(19) United States
(12) Patent Application Publication
(10) Pub. No.: US 2025/0325668 A1
(43) Pub. Date: Oct. 23, 2025

(54) TARGETING COMMON SOMATIC MUTATIONS IN BREAST CANCER WITH NEO-ANTIGEN SPECIFIC ADOPTIVE T CELL THERAPY

(71) Applicant: Baylor College of Medicine, Houston, TX (US)

(72) Inventors: Valentina Hoyos, Houston, TX (US); Paul Shafer, Houston, TX (US); Ann Marie Leen, Houston, TX (US); Wingchi Leung, Houston, TX (US); Premal Lulla, Houston, TX (US); Cliona M. Rooney, Bellaire, TX (US)

(73) Assignee: Baylor College of Medicine, Houston, TX (US)

(21) Appl. No.: 18/698,669

(22) PCT Filed: Oct. 6, 2022
(86) PCT No.: PCT/US2022/077697

§ 371 Date: Apr. 4, 2024

Related U.S. Application Data
(60) Provisional application No. 63/253,651, filed on Oct. 8, 2021.

(51) Int. Cl. A61K 40/42; A61K 40/11; A61K 40/32; C07K 14/725; C12N 5/0783

(52) U.S. Cl. CPC A61K 40/428; A61K 40/11; A61K 40/32; C07K 14/7051; C12N 5/0636; C12N 2501/2302; C12N 2501/515; C12N 2502/30

(57) ABSTRACT

Embodiments of the disclosure concern methods and compositions related to T cell receptors directed against breast cancer neoantigens, including immunotherapeutic compositions of any kind. In specific embodiments, the TCRs are identified following particular methods of producing neoantigen-specific T cells, including particular culturing methods.

This application is a national phase application under 35 U.S.C. § 371 that claims priority to International Application No. PCT/US2022/077697 filed Oct. 6, 2022, which claims priority to U.S. Provisional Patent Application Ser. No. 63/253,651, filed Oct. 8, 2021, both of which are incorporated by reference herein in their entirety.

### INCORPORATION OF SEQUENCE LISTING

The instant application contains a Sequence Listing which has been submitted electronically in XML file format and is hereby incorporated by reference in its entirety. Said XML copy, created on Sep. 30, 2024, is named BAYM_P0348US_SL.xml and is 29,524 bytes in size.

### TECHNICAL FIELD

Embodiments of the disclosure concern at least the fields of cell biology, molecular biology, immunology, and medicine, including cancer medicine.

### BACKGROUND

Adoptive T cell immunotherapies have had extraordinary success treating hematological malignancies but broadening their application toward solid tumors has proven challenging. One requirement for effectively treating solid tumors by immunotherapy is the identification of tumor-specific target antigens. Adoptively transferred T cells that target proteins encoded by somatically mutated genes have had promising results in patients with solid tumors. These mutated proteins represent unique, tumor-restricted sequences that can be recognized by T cells as foreign (i.e., neoantigens) and are thus targeted by neoantigen-specific T cells (Neo-T)¹. The potential of this approach in breast cancer was demonstrated in a patient who experienced complete eradication of her cancer following the adoptive transfer of expanded Neo-T reactive against mutant versions of four proteins found in her cancer². This case demonstrates that targeting somatic mutations unique to a patient's individual tumor can mediate complete and durable tumor regression. However, the report of this case was anecdotal and highly personalized with no data on the overall success rate in a larger group of patients.

The present disclosure satisfies a long-felt need in the art at least to provide effective breast cancer-specific cell immunotherapies and diagnostics.

### SUMMARY OF THE EMBODIMENTS

Embodiments of the disclosure include methods and compositions for the treatment or prevention of cancer. In specific embodiments, the disclosure concerns immunotherapies that utilize adoptive T cells or T cell receptor (TCR) specific for a breast cancer neoantigen associated with the cancer in an individual in need of the therapy, although the neoantigen may also be associated with other types of cancers than breast cancer. The immunotherapies are generated by the hand of man to improve at least one symptom of the cancer, to reduce the severity of one or more symptoms of the cancer, and/or to delay the onset of the cancer or to delay onset or severity of metastasis of the cancer.

The present disclosure provides for development of a safe and potent immunotherapies for breast cancer, although in specific embodiments the therapy is broadly effective because it targets somatic mutations also found in other cancer types, including colon, uterine, cervical, and esophageal cancers. The somatic mutations may be associated with primary and/or metastatic cancers. Twenty-five to 30% of breast cancers harbor one or more specific somatic mutations in hot spot areas of the TP53, PIK3CA, ESR1 and/or AKT1 genes³. Many of these mutations are considered driver mutations and are associated with resistance to conventional therapies^{4,5}. The precision of the cellular immune response allows for a novel, safe and effective therapy targeting the most common breast cancer neoantigens, whether or not by T cells or other immunotherapeutics.

The present disclosure provides neoantigen-specific T cells compositions and methods of using the same to treat or prevent cancer or metastasis of cancer, or to reduce the risk of having cancer or metastasis of cancer. In some embodiments, the present disclosure provides compositions comprising a population of T cells that recognize one or more neoantigens associated with breast and other cancers, including T cells that have been engineered to recognize one or more neoantigens associated with breast and other cancers. The neoantigen-specific T cells comprise TCRs that are directed to the neoantigens, and these TCRs are utilized in immunotherapeutic compositions.

In some embodiments, the neoantigen-specific T cells from which the TCRs are derived are generated in a particular manner, such as by contacting peripheral blood mononuclear cells (PBMCs) with one or more pepmix libraries, each pepmix library containing a plurality of overlapping peptides spanning the neoantigen. In some embodiments, the neoantigen-specific T cells are generated by contacting T cells with antigen presenting cells (APCs) (such as dendritic cells (DCs), B cells, macrophages, lymphoblastoid cell lines, etc.) primed with a plurality of pepmix libraries, each pepmix library containing a plurality of overlapping peptides spanning all or at least a portion of a neoantigen. In some embodiments, the neoantigen-specific T-lymphocytes are generated by contacting T cells with APCs such as DCs nucleofected with at least one DNA plasmid encoding at least one or a portion of one neoantigen.

In some embodiments, the production methods of the T cells include one or more ex vivo culture steps in the presence of two or more of IL-7, IL-12, IL-15, and IL-6. In some embodiments, the neoantigen-specific T cells have expanded sufficiently within about 9 to about 18 days of culture such that they are ready for administration to an individual in need thereof. In cases wherein there are multiple stimulations, the culture may comprise IL-7 and IL-15 and/or IL-2 at a subsequent stimulation, such as a second and/or further stimulation. In specific embodiments, the culture medium comprises 1, 2, 3, 4, 5, or more cytokines, and in other embodiments the culture medium consists essentially of 1, 2, 3, 4, or 5 or more cytokines, and in other embodiments, the culture medium consists of 1, 2, 3, 4, or 5 or more cytokines. In specific embodiments with respect to cytokines only, the culture medium comprises 1, 2, 3, 4, 5, or more cytokines, and in other embodiments the culture medium consists essentially of 1, 2, 3, 4, or 5 or more cytokines, and in other embodiments, the culture medium consists of 1, 2, 3, 4, or 5 or more cytokines: in such cases, the culture medium comprises one or more other components that are effective for cell stimulation and production.

In some embodiments, the present disclosure provides a composition comprising a population that has T cells that recognize one or more breast cancer neoantigens, one or more of which neoantigens may be present on different types of cancer cells that may or may not arise from the same type of primary cancer. In specific cases, the composition comprises a population of T cells some of which recognize a first neoantigen and others of which recognize a second neoantigen, and so on. In specific cases, the neoantigens are from the TP53. PIK3CA. ESR1 or AKT genes. Thus, in some embodiments the composition comprises a population that has T cells that recognize one or more neoantigens from TP53 and also a population that has T cells that recognize one or more neoantigens from one or more of PIK3CA. ESR1 or AKT genes. In some embodiments the composition comprises a population that has T cells that recognize one or more neoantigens from PIK3CA and also a population that has T cells that recognize one or more neoantigens from one or more of TP53, ESR1 or AKT genes. In some embodiments the composition comprises a population that has T cells that recognize a neoantigen from ESR1 and also a population that has T cells that recognize a neoantigen from one or more of TP53, PIK3CA or AKT genes. In some embodiments the composition comprises a population that has T cellsthat recognize a neoantigen from AKT and also a population that has T cells that recognize a neoantigen from one or more of TP53, PIK3CA or ESR1 genes.

In some embodiments, the T cells generated by methods encompassed herein express αβT cell receptors, and the α and β chains of the T cells are utilized in other compositions than the cells produced by the methods. In some embodiments, the T cells comprise effector memory T cells and central memory T cells. In some embodiments, the T-lymphocytes are MHC-restricted.

In some embodiments, the present disclosure provides a method of lysing a target breast cancer or other cancer cell that expresses the neoantigen comprising contacting the target cancer cell with any one of the compositions disclosed herein (e.g., a composition comprising immune cells engineered to be neoantigen-specific or comprising a plurality of populations of immune cells engineered to have specificity for any one or more neoantigens and optionally cancer antigens of any kind). In some embodiments, the contacting occurs in vivo in a subject. In some embodiments, the contacting occurs in vivo via administration of the engineered T cells to a subject.

In some embodiments, the present disclosure provides a method of treating or preventing cancer comprising administering to a subject in need thereof any one or more of the compositions disclosed herein (e.g., a composition comprising a population or a plurality of populations of neoantigen-specific T cells). Thus, in embodiments, the present disclosure provides a method of treating or preventing cancer comprising administering to a subject in need thereof a composition comprising a polyclonal population of T cells that recognize neoantigens, such as one or more neoantigens from one or more of TP53, PIK3CA, ESR1 and/or AKT genes.

In some embodiments, the method comprises administering between 103 to 1010 T cells to the subject. In some embodiments, the subject is immunocompetent or immunocompromised. In some embodiments, the subject has cancer, has pre-cancerous cells, or is at risk for cancer above risk for the general population (e.g., family or personal history, genetic marker(s), tobacco user, environmental exposure to chemicals, excessive sunlight, etc.). In some embodiments, the subject has a malignancy of the breast and may be of any gender.

In some embodiments, the composition is administered to the subject a plurality of times. In some embodiments, the administration of the composition effectively treats or prevents or reduces the risk of a cancer in the subject. In some embodiments, the composition effectively treats or prevents or reduces the risk of cancer of any kind including at least any cancer that comprises a neoantigen associated with one or more of TP53, PIK3CA, ESR1 and/or AKT genes. In some embodiments, the subject is a mammal, including at least a human.

In some embodiments, cancer has been detected in the subject (e.g., prior to treatment). In some embodiments, the subject is over 30 years of age. In some embodiments, the subject is over 35, 40, 45, 50, 55, 60, 65, 70, 75, 80, 85, or 90 years of age. In some embodiments, the subject is under 30 years of age. In some embodiments, the subject is at a higher risk for cancer because of having one or more risk factors.

Embodiments of the disclosure include compositions comprising a population of neoantigen-specific T cells that recognize one or more breast cancer-associated neoantigens. The neoantigen-specific T cells recognize one or more breast cancer-associated neoantigens in TP53, AKT1, ESR1, PIK3CA, ERBB2, FRMPD3, GOLGA6L6, HISTIH2AE, MUC4, NBPF12, or SF3B1 genes. In some cases, the neoantigen is from TP53 and is selected from the group consisting of TP53 R175H: TP53 R248Q: TP53 R248W: TP53 R273C; and TP53 R273H. In some cases, the neoantigen is from ESR1 and is selected from the group consisting of ESR1 K303R: ESR1 Y537S; and ESR1 D538G. In some cases, the neoantigen is from AKT1 and is AKT1 E17K. In some cases, the neoantigen is from PIK3CA and is selected from the group consisting of PIK3CA E542K: PIK3CA E545K: PIK3CA H1047R; and PIK3CA H1047L.

In particular embodiments, the neoantigen-specific T cells are generated by contacting peripheral blood mononuclear cells (PBMCs) with one or more pepmix libraries, each pepmix library comprising a plurality of overlapping peptides spanning all or at least a portion of the neoantigen. The neoantigen-specific T cells may be generated by contacting T cells with antigen presenting cells (APCs, including dendritic cells, B cells, monocytes, etc.) primed with one or a plurality of pepmix libraries, each pepmix library comprising a plurality of overlapping peptides spanning all or at least a portion of the neoantigen or neoantigens. The neoantigen-specific T cells may be generated by contacting T cells with APCs nucleofected with at least one DNA plasmid encoding at least one or a portion of one breast cancer-associated neoantigen. In certain embodiments, the neoantigen-specific T cells are cultured ex vivo in the presence of two or more cytokines selected from the group consisting of IL-7, IL-12, IL-15 and IL-6.

In specific embodiments, the T-cell receptor of the T cells is encoded by DNA as follows: (a) an alpha chain encoded by sequence comprising SEQ ID NO:4 or comprising at least 85% identity to SEQ ID NO:4; and a beta chain encoded by sequence comprising SEQ ID NO:5 or comprising at least 85% identity to SEQ ID NO:5: (b) an alpha chain encoded by sequence comprising SEQ ID NO:6 or comprising at least 85% identity to SEQ ID NO:6; and a beta chain encoded by sequence comprising SEQ ID NO:7 or comprising at least 85% identity to SEQ ID NO: 7: (c) an alpha chain encoded by sequence comprising SEQ ID NO:8 or comprising at least 85% identity to SEQ ID NO:8; and a beta chain encoded by sequence comprising SEQ ID NO:9 or comprising at least 85% identity to SEQ ID NO:9: or (d) an alpha chain encoded by sequence comprising SEQ ID NO: 10 or comprising at least 85% identity to SEQ ID NO:10; and a beta chain encoded by sequence comprising SEQ ID NO: 11 or comprising at least 85% identity to SEQ ID NO: 11. In some embodiments, the T-cell receptor of the T cells comprises: (a) an alpha chain comprising SEQ ID NO: 12 or comprising at least 85% identity to SEQ ID NO: 12; and a beta chain comprising SEQ ID NO: 13 or comprising at least 85% identity to SEQ ID NO: 13: (b) an alpha chain comprising SEQ ID NO: 14 or comprising at least 85% identity to SEQ ID NO: 14; and a beta chain comprising SEQ ID NO: 15 or comprising at least 85% identity to SEQ ID NO:15: (c) an alpha chain comprising SEQ ID NO: 16 or comprising at least 85% identity to SEQ ID NO: 16; and a beta chain comprising SEQ ID NO: 17 or comprising at least 85% identity to SEQ ID NO: 17: or (d) an alpha chain comprising SEQ ID NO:18 or comprising at least 85% identity to SEQ ID NO: 18; and a beta chain comprising SEQ ID NO:19 or comprising at least 85% identity to SEQ ID NO: 19.

Specific embodiments encompass compositions comprising a polyclonal population of neoantigen-specific T cells that recognize one or more breast cancer-associated neoantigens and that recognize one or more other cancer antigens. The one or more other antigens may or may not be breast cancer-associated antigens. In some cases, the one or more other cancer antigens are neoantigens. Any neoantigen herein may be associated with cancer other than breast cancer. In specific cases, the neoantigen-specific T cells recognize one or more breast cancer-associated neoantigens in TP53. AKT1. ESR1. PIK3CA. ERBB2. FRMPD3. GOLGA616. HISTIH2AE. MUC4. NBPF12, or SF3BI genes. Specific neoantigens include AKT1 E17K: ESR1 K303R: ESR1 Y537S: ESR1 D538G: PIK3CA E542K: PIK3CA E545K: PIK3CA H1047R: PIK3CA H1047L: TP53 R175H: TP53 R248Q: TP53 R248W: TP53 R273C: TP53 R273H: ERBB2 L755S: ESR1 E380Q: ESR1 L536P: ESR1 S463P: ESR1 Y537C: ESR1 Y537N: EXOC4 S21L: FRMPD3 Q1757E: GOLGA6L6 M4721: HISTIH2AE K128M: MUC4 S2858P: NBPF12 E125Q: PIK3CA E453K: PIK3CA E726K: PIK3CA N345K: PIK3CA Q546K: SF3B1 K700E: TP53 G245S: TP53 H179R: TP53 H193R: TP53 1195T: TP53 R282W: or TP53 Y220C.

In particular embodiments, the neoantigen-specific T cells are generated by contacting PBMCs with a plurality of pepmix libraries, each pepmix library containing a plurality of overlapping peptides spanning all or a portion of a breast cancer-associated neoantigen or one or more other cancer antigens. The neoantigen-specific T cells may be generated by contacting T cells with APCs primed with a plurality of pepmix libraries, each pepmix library containing a plurality of overlapping peptides spanning all or a portion of a breast cancer-associated neoantigen, wherein at least one of the plurality of pepmix libraries spans a breast cancer-associated neoantigen and wherein at least one additional pepmix library of the plurality of pepmix libraries spans one or more other cancer antigens. The neoantigen-specific T cells may be generated by contacting T cells with APCs nucleofected with at least one DNA plasmid encoding at least one breast cancer-associated neoantigen and at least one DNA plasmid encoding one or more other cancer antigens. In specific cases, the population comprises CD4+ T-lymphocytes and CD8+ T-lymphocytes. The neoantigen-specific T cells may express αβT cell receptors. The composition may comprise MHC-restricted neoantigen-specific T cells. In at least some cases, the neoantigen-specific T cells comprise central memory and effector memory T cells. The neoantigen-specific T cells may be cultured ex vivo in the presence of two or more cytokines selected from the group consisting of IL-7, IL-12, IL-15 and IL-6. In specific cases, the neoantigen-specific T cells produce effector cytokines/molecules including IFN-gamma, TNF-alpha, GM-CSF, Granzyme-B, or perforin upon exposure to antigen, and the neoantigen-specific T cells may be able to lyse neoantigen-expressing target cells. The neoantigen-specific T cells do not significantly lyse non-cancerous autologous or allogenic target cells, in specific embodiments.

Embodiments of the disclosure include pharmaceutical compositions comprising any cells or other molecules encompassed herein, and any pharmaceutical composition may be formulated for intravenous delivery.

In specific embodiments, there is a composition comprising a chain and/or b chain T-cell receptor (TCR) polypeptides from any cells encompassed herein, and the α chain and/or b chain T-cell receptor (TCR) polypeptides may or may not be labeled, or in some cases another molecule in the composition is labeled. The label may be fluorescent, colorimetric, photochromic, radioactive, is an Fc fragment of a human or non-human immunoglobulin, or is an enzyme that is activateable or deactivatiable upon contact with the neoantigen. In specific cases, the α chain and/or b chain T-cell receptor (TCR) polypeptides are comprised in a transgenic molecule, such as a chimeric antigen receptor, a multi-specific T cell engager, or wherein the α chain and/or b chain T-cell receptor (TCR) polypeptides are conjugated to a drug (e.g., breast cancer chemotherapeutic) and/or radiotherapeutic molecule.

The disclosure encompasses vectors comprising a chain and/or b chain T-cell receptor (TCR) polypeptides from any cells encompassed herein. Embodiments include cells engineered to express a chain and/or b chain T-cell receptor (TCR) polypeptides from any cells encompassed herein, including immune cells, such as T cell, natural killer cell, natural killer T cell, B cell, or macrophage. The cells may be engineered to comprise one or more further modifications, such as (a) disruption of one or more endogenous genes of the cells: (b) one or more non-natural antigen-specific T cell receptors other than the engineered receptor comprising the α chain and/or b chain T-cell receptor (TCR) sequences: (c) one or more cytokine receptors: (d) one or more chimeric cytokine receptors: (e) one or more cytokines: or (f) a combination thereof. In some cases, the non-natural T cell receptors of (c) are TCRs directed against a tumor-associated antigen. In specific cases, the neoantigen-specific T cells are modified to be directed against a tumor-associated antigen.

Embodiments of the disclosure include methods of lysing a target cells comprising contacting the target cell with any composition, including pharmaceutical composition and including cells, encompassed herein. In specific embodiments, contacting occurs in vivo in a subject. In specific embodiments, the contacting occurs in vivo via administration of the neoantigen-specific T-lymphocytes to a subject.

Embodiments of the disclosure include methods of treating or preventing breast cancer in an individual in need thereof, comprising administering to the individual any composition, including pharmaceutical composition, and including cells encompassed herein. In specific embodiments, between 5×10⁶ and 5×10⁷ engineered or otherwise neoantigen-specific T-lymphocytes/m² are administered to the individual. The composition may be administered to the individual a plurality of times. The individual is a mammal, including a human, in specific embodiments.

Embodiments of the disclosure include methods of treating or preventing breast cancer in an individual, comprising administering to the individual an effective amount of a population of neoantigen-specific T cells, wherein the neoantigen-specific T cells comprise a T cell receptor selected based on the ability to selectively bind a neoantigen in the cancer of the individual and wherein the neoantigen is HLA matched to the individual. In specific embodiments, the neoantigen-specific T cells are from a library of cell lines of neoantigen-specific T cells, wherein the T cell receptor is directed to a neoantigen that is known and the specific HLA polymorphism that presents the peptide to which the TCR binds is also known. The neoantigen-specific T cells may be autologous or allogeneic with respect to the individual.

Embodiments of the disclosure include methods of producing neoantigen-specific T cells for treating or preventing breast cancer in an individual, comprising the steps of: (a) obtaining or generating a library of cell lines of neoantigen-specific T cells, said step comprising one of the following: (1) contacting PBMCs with one or more pepmix libraries, each pepmix library containing a plurality of overlapping peptides spanning all or at least a portion of one or more breast cancer-associated neoantigens: (2) contacting T cells with APCs such as dendritic cells (DCs) primed with a plurality of pepmix libraries, each pepmix library containing a plurality of overlapping peptides spanning all or at least a portion of one or more breast cancer-associated neoantigens: or (3) contacting T cells with APCs such as DCs nucleofected with at least one DNA plasmid encoding at least one breast cancer-associated neoantigen, or a portion thereof: (b) screening cell lines from the library to select for cells that demonstrate specificity for the neoantigen, but not specificity for a corresponding wild-type sequence, and also identifying or having known HLA class restriction for the neoantigen: (c) transfecting or transducing T cells with vectors expressing transgenic T-cell receptors from the cells in (b) that demonstrate specificity for the neoantigen; and (d) administering to the individual an effective amount of the transduced or transfected T cells from (c) when the cancer of the individual comprises the neoantigen and when the individual is HLA matched for the neoantigen.

Embodiments of the disclosure include methods of treating or preventing breast cancer in an individual, comprising administering to the individual an effective amount of a population of neoantigen-specific T cells, wherein said T cells are generated by the following: (a) producing a library of cell lines of neoantigen-specific T cells, said producing step comprising one of the following: (1) contacting PBMCs with one or more pepmix libraries, each pepmix library containing a plurality of overlapping peptides spanning all or at least a portion of one or more breast cancer-associated neoantigens: (2) contacting T cells with APCs such as dendritic cells (DCs) primed with a plurality of pepmix libraries, each pepmix library containing a plurality of overlapping peptides spanning all or at least a portion of one or more breast cancer-associated neoantigens: or (3) contacting T cells with APCs such as DCs nucleofected with at least one DNA plasmid encoding at least one breast cancer-associated neoantigen, or a portion thereof: (b) screening cell lines from the library to select for cells that demonstrate specificity for the neoantigen, but not a corresponding wild-type sequence, and also identifying HLA class restriction for the neoantigen: (c) administering to the individual an effective amount of cells from the cell line when the cancer of the individual comprises the neoantigen and when the individual is HLA matched for the neoantigen. In specific embodiments, the neoantigen-specific T cells are cultured ex vivo in the presence of two or more cytokines selected from the group consisting of IL-7, IL-12. IL-15 and IL-6.

Specific embodiments include an engineered T cell receptor (TCR) comprising sequence encoded by DNA as follows: (a) an alpha chain encoded by sequence comprising SEQ ID NO:4 or comprising at least 85% identity to SEQ ID NO:4; and a beta chain encoded by sequence comprising SEQ ID NO:5 or comprising at least 85% identity to SEQ ID NO:5: (b) an alpha chain encoded by sequence comprising SEQ ID NO:6 or comprising at least 85% identity to SEQ ID NO: 6; and a beta chain encoded by sequence comprising SEQ ID NO:7 or comprising at least 85% identity to SEQ ID NO:7: (c) an alpha chain encoded by sequence comprising SEQ ID NO:8 or comprising at least 85% identity to SEQ ID NO:8; and a beta chain encoded by sequence comprising SEQ ID NO:9 or comprising at least 85% identity to SEQ ID NO:9: or (d) an alpha chain encoded by sequence comprising SEQ ID NO: 10 or comprising at least 85% identity to SEQ ID NO: 10; and a beta chain encoded by sequence comprising SEQ ID NO: 11 or comprising at least 85% identity to SEQ ID NO:11.

Specific embodiments, include an engineered T cell receptor (TCR) comprising: (a) an alpha chain comprising SEQ ID NO: 12 or comprising at least 85% identity to SEQ ID NO: 12; and a beta chain comprising SEQ ID NO: 13 or comprising at least 85% identity to SEQ ID NO: 13: (b) an alpha chain comprising SEQ ID NO: 14 or comprising at least 85% identity to SEQ ID NO: 14; and a beta chain comprising SEQ ID NO: 15 or comprising at least 85% identity to SEQ ID NO: 15: (c) an alpha chain comprising SEQ ID NO:16 or comprising at least 85% identity to SEQ ID NO: 16; and a beta chain comprising SEQ ID NO:17 or comprising at least 85% identity to SEQ ID NO: 17: or (d) an alpha chain comprising SEQ ID NO: 18 or comprising at least 85% identity to SEQ ID NO:18; and a beta chain comprising SEQ ID NO: 19 or comprising at least 85% identity to SEQ ID NO: 19.

In particular embodiments, the disclosure includes cells comprising one or more TCRs as encompassed herein, and the cells may be any kind of immune cell, including at least a T cell, natural killer cell, natural killer T cell, B cell, or macrophage.

### BRIEF DESCRIPTION OF THE DRAWINGS

FIGS. 1A-IC. Neoantigen-specificity of Neo-T cell lines. (FIG. 1A) IFNγ ELISpot was used to determine the specificity of Neo-T cell lines for each individual overlapping peptide. The inventors have generated 14 Neo-T cell lines from healthy donors and 11 from patients whose tumors express a targeted driver mutation (indicated by bold borders). (FIG. 1B) IFNγ ELISpot results confirm reactivity of HD #1's Neo-T cell line against ESR1 Y537S peptide 4 and D538G peptide 3 and not the corresponding wild type peptides. (FIG. 1C) Representative IFNγ ELISpot results from HD #2 ESR1 Y537S neo-peptide 5 reactivity in the presence or absence of HLA blocking antibodies (HLA class I clone W6/32 or class II clone Tu39). Results confirm reactivity is HLA class I restricted.

FIG. 2. Cr⁵¹ release assay showing HD #1 Neo-T cells kill autologous PHA blasts pulsed with ESR1 Y537S peptide 4 and D538G peptide 3 and not the WT peptide. ESR1 WT is SEQ ID NO: 1: ESR1 Y537S is SEQ ID NO:2; and ESR1 D538G is SEQ ID NO:3.

FIGS. 3A-3B. (FIG. 3A) The Donor 1 Neo-T line was stimulated for 12 hrs with ESR1 WT or mutant peptides and then stained with barcoded ‘hashtag’ antibodies, pooled, and submitted for scRNAseq. ESR1 WT is SEQ ID NO:1: ESR1 Y537G is SEQ ID NO:2; and ESR1 D538G is SEQ ID NO: 3. (FIG. 3B) Resulting expression of IFN-γ and TNF-α by the Donor 1 Neo-T line in response to ESR1 WT or mutant peptide stimulation. Two identified neoantigen-specific T cell clonotypes, clones 16 and 21, are indicated.

FIGS. 4A-4E. (FIG. 4A) Schema of the SFG.neo-TCR retroviral vector. (FIG. 4B) T cells transduced with the TP53 R248W neo-TCRs identified from HD #13 Neo-T cell line. Expression of the Neo-TCR measured by flow cytometry. (FIG. 4C) IFNγ ELISpot assay confirming specificity of the neo-TCR+ T cells against the TP53 R248W neo-peptide. (FIG. 4D) 6 hr Cr⁵¹ release cytotoxicity assay showing Neo-TCR+ T cells kill autologous PHA blasts pulsed with the neo-peptide and not the WT. (FIG. 4E) Donor 13 HLA class I alleles were cloned from cDNA and subsequently subcloned into an SFG retroviral vector and individual transduced into allogeneic T cells. Autologous or HLA-transduced allogeneic T cells were cocultured with allogeneneic TP53R248W-TCR transduced T cells in the presence of TP53R248W-neopeptide in an ELISpot assay.

FIGS. 5A-5E. (FIG. 5A) Transduction efficiency of recipient T cells transduced to express the clonotype 21 TCR as assessed by flow detection of the murine TCR beta constant chain. (FIG. 5B) Specificity of clonotype 21 TCR transduced T cells (Dual.ERneo.21.Ts) for ESR1m mutations assessed by IFN-γ ELISpot. (FIG. 5C) Cr-51 release cytotoxicity assay whereby Cr-stained autologous PHA blasts were pulsed with ESR1 WT or mutant peptides and cultured with Dual.ERneo.21.Ts for 6 hrs. (FIG. 5D) The ESR1 Y537S minimal epitope recognized by Dual. ERneo.21.Ts was determined through an IFN-γ ELISpot assay whereby Dual. ERneo.21.Ts were stimulated with the 15 mer Y537S peptide or various 10′ and 9′mer peptides that contain the mutated amino acid. From top to bottom in order, the peptides are SEQ ID NO:2 and SEQ ID NOs: 20-28. (FIG. 5E) Donor 1 HLA class I alleles were cloned from cDNA and subsequently subcloned into an SFG retroviral vector and individual transduced into allogeneic T cells. HLA-transduced allogeneic T cells were pulsed with ESR1m peptides and Cr-stained and cocultured with Dual. ERneo.21. Ts for 6 hrs.

FIGS. 6A-6D. (FIG. 6A) Transduction efficiency of recipient T cells transduced to express the clonotype 16 TCR as assessed by flow detection of the murine TCR beta constant chain. (FIG. 6B) Specificity of clonotype 16 TCR transduced T cells (ERneo. 16.Ts) for ESR1 Y537S assessed by IFN-γ ELISpot. (FIG. 6C) Donor 1 HLA class I alleles were cloned from cDNA and subsequently subcloned into an SFG retroviral vector and individual transduced into allogeneic T cells. Autologous or HLA-transduced allogeneic T cells were cocultured with allogeneneic ERneo. 16.Ts in the presence of ESR1Y537S-peptide in an IFN-γ ELISpot assay. (FIG. 6D) The ESR1 Y537S minimal epitope recognized by ERneo. 16.Ts was determined through an IFN-γ ELISpot assay whereby ERneo. 16.Ts were stimulated with the 15 mer Y537S peptide or various 10′ and 9′mer peptides that contain the mutated amino acid. From top to bottom in order, the peptides are SEQ ID NO:2 and SEQ ID NOs: 20-28.

### DETAILED DESCRIPTION

### Definitions

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The term “about” when immediately preceding a numerical value means±0% to 10% of the numerical value, ±0% to 10%, ±0% to 9%, ±0% to 8%, ±0% to 7%, ±0% to 6%, ±0% to 5%, ±0% to 4%, ±0% to 3%, ±0% to 2%, ±0% to 1%, ±0% to less than 1%, or any other value or range of values therein. For example, “about 40” means±0% to 10% of 40 (i.e., from 36 to 44).

The use of the term “or” in the claims is used to mean “and/or” unless explicitly indicated to refer to alternatives only or the alternatives are mutually exclusive, although the disclosure supports a definition that refers to only alternatives and “and/or.”

The term “neoantigen” as used herein refers to somatic mutations expressed only by cancer cells, including at least solid tumor cells, such as breast cancer cells or other cancer cells.

The term “neoantigen-specific T-lymphocytes” or “neoantigen-specific T cell lines” or “neoantigen-specific T cells” or “neo-specific T cells” are used interchangeably herein to refer to T cell lines that have specificity and potency against a cancer neoantigen or cancer neoantigens of interest or a cancer neo-epitope.

The term “neo-epitope” as used herein refers to a peptide harboring a mutated amino acid/nucleotide sequence capable of binding to class I and/or class II HLA molecules and being recognized by the T cell receptor of natural or engineered T cells.

The term “driver mutation” as used herein refers to a mutation that confers a growth advantage on cancer cells, thereby driving positive selection within the cancer cell population.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. With respect to cells, the cells may be engineered because they express one or more heterologous genes (such as synthetic TCRs, receptors of any kind including antigen receptors, and/or cytokines) and/or the cells are engineered by having reduced expression of one or more endogenous genes, all of which case(s) the engineering is performed by the hand of man. With respect to an antigen receptor, including a TCR, the antigen receptor may be considered engineered because it comprises multiple components that are genetically recombined to be configured in a manner that is not found in nature, such as in the form of a fusion protein of components not found in nature so configured.

As used herein, the terms “individual, “subject,” and “patient,” are used interchangeably herein and generally refers to an individual in need of treatment. The subject can be any animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having cancer. The subject may be undergoing or having undergone cancer treatment. The “subject” or “individual,” as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants. The individual may be of any gender or race or ethnicity.

The terms “treat”, “treating”, “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the disease, condition, or disorder. In some instances, treatment is curative or ameliorating.

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

The term “heterologous” as used herein refers to being derived from a different cell type or a different species than the recipient. In specific cases, it refers to a gene or protein that is synthetic and/or not from a natural T cell. The term also refers to synthetically derived genes or gene constructs.

The terms “administering”, “administer”, “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

As used herein, the terms “comprise,” “comprising,” “includes,” “including,” “has,” “having,” “contains,” “containing,” “characterized by,” or any other variation thereof, are intended to encompass a non-exclusive inclusion, subject to any limitation explicitly indicated otherwise, of the recited components. For example, a composition and/or method that “comprises” a list of elements (e.g., components or features or steps) is not necessarily limited to only those elements (or components or features or steps), but may include other elements (or components or features or steps) not expressly listed or inherent to the composition and/or method.

As used herein, the phrases “consists of” and “consisting of” exclude any element, step, or component not specified. For example, “consist of” or “consisting of” used in a claim would limit the claim to the components, materials or steps specifically recited in the claim except for impurities ordinarily associated with therewith (i.e., impurities within a given component). When the phrase “consist of” or “consisting of”′ appears in a clause of the body of a claim, rather than immediately following the preamble, the phrase “consist of” or “consisting of′ limits only the elements (or components or steps) set forth in that clause: other elements (or components) are not excluded from the claim as a whole.

Other objects, feature and advantages of the present invention will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

The following discussion is directed to various embodiments of the disclosure. The term “invention” is not intended to refer to any particular embodiment or otherwise limit the scope of the disclosure. Although one or more of these embodiments may be particularly considered, the embodiments disclosed should not be interpreted, or otherwise used, as limiting the scope of the disclosure, including the claims. In addition, one skilled in the art will understand that the following description has broad applications, and the discussion of any embodiment is meant only to be exemplary of that embodiment, and not intended to intimate that the scope of the disclosure, including the claims, is limited to that embodiment.

The present disclosure provides cancer therapy by the administration of one or more immunotherapeutic compositions of any kind that utilize T cell receptors (TCRs) obtained from methods disclosed herein of producing breast cancer neoantigen-specific T cells. The immunotherapeutic compositions control cancer cell growth, including to kill cancer cells. In specific embodiments, immune cells are engineered to express the TCRs, and without wishing to be bound by any theories, the engineered cells recognize and kill neoantigen-bearing cancer cells via the engineered TCR that binds to major histocompatibility complex (MHC) molecules expressed on target cells that present neoantigen-derived peptides. Thus, in some embodiments, the neoantigen-specific T cells disclosed herein are useful for treating or preventing or reducing the risk of cancers that bear or express the neoantigen. In specific embodiments, the neoantigen associated with the cancer cells is expressed on the surface of the cancer cells. In other embodiments, the TCRs obtained from the breast cancer neoantigen-specific T cells are utilized in immunotherapeutic compositions other than cells themselves, such as in receptor molecules or multi-specific T-cell engagers, for example.

In some embodiments, the T cells are produced from mononuclear cells (MNCs) procured from healthy donors, which may or may not be from a partially HLA-matched “off-the-shelf” product. In some embodiments, the T cells are produced from peripheral blood mononuclear cells (PBMCs) that may or may not be procured from healthy, pre-screened donors, which may be available as a partially HLA-matched “off-the-shelf” product. The PBMCs may or may not come from the individual in need of cancer treatment. In some embodiments, the donor may be a person who has already had cancer, and in some cases the person has cancer in which at least one neoantigen is in common with the individual in need of cancer treatment.

In some embodiments, the engineered T cells and the TCRs themselves as described herein respond to (or “are specific for” or “are directed to”) at least one neoantigen. In some embodiments, the engineered T cells as described herein may be multi-neoantigen specific T cells that respond to a first neoantigen and one or more additional neoantigens. In some embodiments, the T cells as described herein may respond to a first neoantigen associated with breast cancer and one or more additional neoantigens that are also associated with breast cancer. The cancer for which the neoantigens are associated may or may not be metastatic breast cancer. In some embodiments, the T cells described herein respond at least to a neoantigen from the TP53 gene. In some embodiments, the T cells described herein respond at least to a neoantigen from the PIK3CA gene. In some embodiments, the T cells described herein respond at least to a neoantigen from the ESR1 gene. In some embodiments, the T cells described herein respond at least to a neoantigen from the AKT gene.

In some embodiments, the engineered T cells disclosed herein that are specific for a breast cancer neoantigen also display reactivity against the same neoantigen in another type of cancer that is not breast cancer, such as those wherein the cancer displays the same neoantigen. Thus, while the neoantigen-specific T cells disclosed herein may in some embodiments be utilized for treating or preventing breast cancer, they may additionally or alternatively be utilized for treating other cancers, in some embodiments. For example, in one embodiment the neoantigen-specific T cells disclosed herein are used to treat or prevent another type of cancer, such as uterine, esophageal, cervical lung, colon, brain, liver, kidney, skin, stomach, ovarian, cervical, testicular, endometrial, blood, and so forth.

### I. Breast Cancer Neoantigens

The present disclosure provides cell and other immunotherapies that target somatic mutations expressed only by cancer cells, otherwise known as neoantigens. In particular embodiments, the mutations are of any kind, including those that are the following: (1) driver mutations (i.e., mutations that cause cells to become cancer cells): (2) “hot spot” mutations (i.e., mutations that are frequently detected within one cancer type, or across many different diseases/tumor types and that occur more frequently than expected compared to a background frequency; and/or (3) a patient-specific mutations.

The nature of the mutation(s) for the neoantigen may be of any kind of mutation, including a substitution, deletion, insertion, missense, nonsense, translocation, or frameshift, as examples. In some cases, the neoantigen comprises a neo-epitope that encompasses sequence surrounding the specific mutation. For example, there may be a single substitution at one amino acid, but the neoantigen comprises a neo-epitope of one or several amino acids on the N-terminal and/or C-terminal side of the single substitution.

The breast cancer neoantigen may be associated with one or more other types of cancers. For example, the neoantigen may be identified in a certain percentage of individuals with a first type of cancer and also associated in a lesser percentage of individuals with a second or third, etc., type of cancer. In some cases, the neoantigen is only identified in one particular type of cancer. Samples from an individual identified as having a certain type of cancer may be assayed for the presence of any type of neoantigen prior to determining whether or not the individual should be administered therapies encompassed herein.

The neoantigen may be associated with one or more particular genes, and those one or more genes may or may not be known to be prone to having cancer-causing mutations, such as those affiliated with a certain type of cancer. In certain embodiments, more than one neoantigen is known to be present in one or more genes for a particular type of cancer, and the individual may or may not be subject to methods of determining whether or not the more than one neoantigen is present in the cells of the individual.

In some embodiments, the neoantigens to which the TCRs are directed may be as follows: AKT1 E17K: ESR1 K303R: ESR1 Y537S: ESR1 D538G: PIK3CA E542K: PIK3CA E545K: PIK3CA H1047R: PIK3CA H1047L: TP53 R175H: TP53 R248Q: TP53 R248W: TP53 R273C: TP53 R273H: ERBB2 L755S: ESR1 E380Q: ESR1 L536P: ESR1 S463P: ESR1 Y537C: ESR1 Y537N: EXOC4 S21L: FRMPD3 Q1757E: GOLGA6L6 M472I: HIST1H2AE K128M: MUC4 S2858P: NBPF12 E125Q: PIK3CA E453K: PIK3CA E726K: PIK3CA N345K: PIK3CA Q546K: SF3B1 K700E: TP53 G245S: TP53 H179R: TP53 H193R: TP53 1195T: TP53 R282W: or TP53 Y220C.

In specific embodiments, one or more mutations in ATK1, PIKCA, and/or TP53 are associated with breast cancer but are also common in uterine and colon cancer. In some embodiments, AKT1 and PIK3CA mutations are associated with breast cancer but are also common in cervical cancer. In some embodiments, TP53 mutations are associated with breast cancer but are also common in esophageal cancer.

### II. Neoantigen Peptide Library

Methods of the disclosure utilize neopeptide libraries as a means to produce neoantigen-specific T cells having T-cell receptors (TCRs) that recognize at least one neopeptide representing a neoantigen. Neoantigen peptide libraries are a collection of peptides that represent a particular neoantigen. The neoantigen comprises at least one mutation, and the mutation may or may not be located at different positions within the collection of peptides. A given mutation may be successively staggered among a collection of peptides. Among a collection of peptides, a given mutation may be present on a first peptide at a first position, present on a second peptide at a second position that is adjacent to the first position, present on a third peptide at a third position that is adjacent to the second position, present on a fourth peptide at a fourth position that is adjacent to the third position, and so on. The mutation may be at or towards one end of the peptide, or the mutation may be generally centrally located within the peptide. In a specific case, a single peptide (e.g., 25-30 amino acids in length) is generated and the mutation is generally centrally located within the peptide. The library comprises a plurality of peptides at least some of which are non-identical, in specific cases, and such a library may be referred to as a pepmix. In some cases, a pepmix comprises two or more libraries, such as when each of the libraries comprise a collection of pepmixes that are directed to different neoantigens, and at least some of the peptides are non-identical within the libraries.

The peptides may be said to represent a particular neoantigen by each of the peptides comprising sequence of at least some of the neoantigen. Collectively, the pepmix may comprise the entire region of the neoantigen such that each peptide within a pepmix comprises the mutated region at a different location. In particular embodiments, each of the peptides comprise a specific mutation sequence of the neoantigen. Some of the peptides may comprise the mutation sequence at or towards an N-terminal end of the peptide, at or towards a C-terminal end of the peptide, between the center and the end of the peptide, or approximately in the center of the peptide. In specific cases, each mutated neoantigen sequence of a given library may be located at different positions among the individual peptides, although some of the peptides in a given library may be identical or substantially identical (e.g., greater than about 75, 80, 85, 90, or 95%) to one another.

Pepmixes utilized in the disclosure may or may not be from commercially available peptide libraries. The peptides may be 15 amino acids long, or about 15 amino acids long, and they may overlap one another, such as by 11 amino acids, or by about 11 amino acids, in certain aspects. In some cases, they may be generated synthetically. Examples include those from JPT Technologies (Springfield, VA) or Miltenyi Biotec (Auburn, CA). In particular embodiments, the neoantigen peptides are at least (about) 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least (about) 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example.

In some embodiments, the amino acids as used in the pepmixes have at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99, at least 99.9% purity, inclusive of all ranges and subranges therebetween. In some embodiments, the amino acids as used here in the pepmixes have at least 70% purity.

The mixture of different peptides may include any ratio of the different peptides, although in some embodiments each particular peptide is present at substantially the same numbers in the mixture as another particular peptide. The methods of preparing and producing pepmixes for multiviral cytotoxic T cells with broad specificity is described in US2018/0187152, which is incorporated by reference in its entirety.

### III. Generation and Use of Neoantigen-Specific TCRs

The disclosure encompasses methods of producing neoantigen-specific T cell therapy using methods that are enhanced compared to known methods. The methods generate neoantigen-specific T cells that target at least one neoantigen, including 1, 2, 3, 4, 5, or more neoantigens. Following their generation, in specific embodiments the TCR identity is determined and further utilized.

In particular embodiments, the present disclosure concerns methods and compositions in which T cells are engineered to express TCRs directed against a particular neoantigen and for which there is HLA matching with respect to a recipient for the cells. The cell therapy may be autologous or allogeneic with respect to an individual in need of the cells, so long as the therapy is neoantigen-specific and HLA matched at least partially. In some cases, TCRs are utilized that can target common neoantigens and that are restricted to HLA alleles present in a broadly representative population.

The disclosure provides for particular methods for producing neoantigen-specific T cells, and in specific embodiments these cells are not themselves utilized as therapy, although in alternative cases they may be utilized as therapy. Instead, following their production, the TCRs that reside in the neoantigen-specific T cells are used as at least part of a therapy.

In some embodiments, methods of producing the T cells comprise isolating mononuclear cells (MNCs), or having MNCs isolated, from blood obtained from donors or from the patient. In some embodiments, the MNCs are PBMCs. MNCs and PBMCs are isolated by using the methods known by a skilled person in the art. By way of example, density centrifugation (gradient) (Ficoll-Paque) can be used for isolating PBMCs. In other examples, cell preparation tubes (CPTs) and SepMate tubes with freshly collected blood can be used for isolating PBMCs.

In some embodiments, the MNCs are PBMCs. By way of example, PBMC can comprise lymphocytes, monocytes, and dendritic cells. By way of example, lymphocytes can include T cells, B cells, and NK cells. In some embodiments, the MNCs as used herein are cultured or cryopreserved. In some embodiments, the process of culturing or cryopreserving the cells can include contacting the cells in culture with one or more neoantigens under suitable culture conditions to stimulate and expand neoantigen-specific T cells.

In some embodiments, contacting the MNCs or PBMCs with one or more neoantigens, or one or more epitopes from one or more neoantigens, stimulates and expands a population of neoantigen-specific T cells from each of the respective donor's MNCs or PMBCs. In some embodiments, the neoantigen-specific T cell lines can be cryopreserved. In some embodiments, the process of culturing or cryopreserving the cells can include contacting the cells in culture with one or more epitopes from one or more neoantigens under suitable culture conditions.

In some embodiments, the culturing of the PBMCs or MNCs is in a vessel comprising a gas permeable culture surface. In one embodiment, the vessel is an infusion bag with a gas permeable portion or a rigid vessel. In one embodiment, the vessel is a G-Rex bioreactor. In one embodiment, the vessel can be any container, bioreactor, or the like, that are suitable for culturing the PBMCs or MNCs as described herein.

In some embodiments, the PBMCs or MNCs are cultured in the presence of one or more cytokines. In some embodiments, the cytokine is two or more cytokines selected from the group consisting of IL-7, IL-12, IL-15 and IL-6. In the method, wherein multiple culturing steps are utilized, the cytokine(s) may be different than in other steps of the method. In one case, the cytokine is IL-7, IL-15, and/or IL-2, such as IL-7 and one or both of IL-15 and IL-2.

In some embodiments, culturing the MNCs or PBMCs can be in the presence of at least 2, 3, 4, 5, 6, 7, 8, 9, 10, 11, 12, 13, 14, 15, 16, 17, 18, 19, 20, or more different pepmixes. Pepmixes, a plurality of peptides, comprise a series of overlapping peptides spanning part of or the entire sequence of a neoantigen. In some embodiments, the MNCs or PBMCs can be cultured in the presence of a plurality of pepmixes. In this instance, each pepmix covers at least one neoantigen that is different than the neoantigen covered by each of the other pepmixes in the plurality of pepmixes. In some embodiments, at least 2, 3, 4, 5, 6, 7, 8, 9, 10, 11, 12, 13, 14, 15, 16, 17, 18, 19, 20 or more different neoantigens are covered by the plurality of pepmixes. In some embodiments, at least one neoantigen from a gene product is covered by the plurality of pepmixes.

In some embodiments, the neoantigen in the one or more pepmixes comprises one or more neoantigens of a gene selected from the group consisting of TP53, PIK3CA, ESR1, and AKT. In certain embodiments, the present disclosure provides T cells comprising or consisting essentially of or consisting of a plurality of T cells directed to a neoantigen in one or more of TP53, PIK3CA, ESR1, and AKT. In embodiments, the present disclosure provides T cells generated by culturing PBMCs in the presence of neoantigens from TP53, PIK3CA, ESR1, and/or AKT. In embodiments, the present disclosure provides T cells (e.g., T cells compositions) comprising a plurality of T cells directed to one or more neoantigens from TP53, PIK3CA, ESR1, and/or AKT. In embodiments, the present disclosure provides T cells (e.g., T cells) consisting essentially of a plurality of T cells directed to the neoantigens from TP53, PIK3CA, ESR1, and/or AKT. The present disclosure also provides multi-T cells comprising a plurality of T cells directed to one or more of the above-mentioned neoantigens and directed to one or more additional antigens. In some embodiments, the neoantigen specific T cells are tested for neoantigen-specific cytotoxicity.

In some embodiments, the T cells in the compositions can be generated by contacting PBMCs with a plurality of pepmix libraries. In some embodiments, each pepmix library contains a plurality of overlapping peptides spanning at least a portion of a neoantigen. In some embodiments, at least one of the plurality of pepmix libraries spans a first antigen from TP53. In some embodiments, at least one additional pepmix library of the plurality of pepmix libraries spans a second antigen, such as a second antigen from PIK3CA, ESR1, and/or AKT. In some embodiments, at least one of the plurality of pepmix libraries spans a first neoantigen from PIK3CA. In some embodiments, at least one additional pepmix library of the plurality of pepmix libraries spans a second antigen, such as a second neoantigen from TP53, ESR1, and/or AKT. In some embodiments, at least one of the plurality of pepmix libraries spans a first neoantigen from ESR1. In some embodiments, at least one additional pepmix library of the plurality of pepmix libraries spans a second neoantigen, such as a second neoantigen from TP53, PIK3CA, and/or AKT. In some embodiments, at least one of the plurality of pepmix libraries spans a first neoantigen from AKT. In some embodiments, at least one additional pepmix library of the plurality of pepmix libraries spans a second neoantigen, such as a second neoantigen from TP53, PIK3CA, and/or ESR1.

In some embodiments, the T cells can be generated by contacting T cells with APCs such as dendritic cells (DCs) nucleofected with at least one DNA plasmid. In some embodiments, the DNA plasmid can encode any neoantigen. In some embodiments, the at least one DNA plasmid may encode a second neoantigen. In some embodiments, the compositions as described herein comprise CD4+ T-lymphocytes and CD8+ T-lymphocytes. In some embodiments, the compositions comprise T cells expressing αβ T cell receptors. In some embodiments, the compositions comprise MHC-restricted CTLs.

In some embodiments, the T-lymphocytes can be cultured ex vivo in the presence of both IL-2 and IL-15. In some embodiments, the multi-neo antigen T cells have expanded sufficiently within 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, or 40 or more days inclusive of all ranges and subranges there between, of culture such that they are ready for administration to a patient. In some embodiments, the multi-neo antigen T cells have expanded sufficiently within any number of days that are suitable for the compositions ad described herein.

The present disclosure provides compositions comprising T cells that exhibit negligible alloreactivity. In some embodiments, the compositions are not cultured in the presence of both IL-7 and IL-4. In some embodiments, the compositions comprising CTL exhibit viability of greater than 70% (e.g., viability of at least about 70%, at least about 75%, at least about 80%, at least about 85%, at least about 90%, or at least about 95%).

In some embodiments, the pepmixes used for constructing the T-lymphocytes are chemically synthesized. In some embodiments, the pepmixes are optionally>10%, >20%, >30%, >40%, >50%, >60%, >70%, >80%, >90%, inclusive of all ranges and subranges there between, pure. In some embodiments, the pepmixes are optionally>90% pure.

In some embodiments, the T cells generated from the methods produce IFNγ and/or TNFα. In some embodiments, the majority of T-lymphocytes that produce IFNγ also produce TNFα. In some embodiments, the T-lymphocytes produce one or more effector molecules selected from IL-2, Granzyme B, IFNγ, TNFα, MIP-1α, and perforin. In some embodiments, the T-lymphocytes produce one or more chemoattractive molecule, e.g., MIP-1β. In some embodiments, the T cells are polyfunctional. As used herein, the term “polyfunctional” refers to T cells that are capable of more than one effector function. Effector functions include production of one or more cytokines and/or chemokines, and lysis of target cells, For example, in some embodiments, the T-lymphocytes produce two or more (e.g., 3, 4, 5, or more) different effector molecules.

In some embodiments, the T cells are able to lyse neoantigen-expressing target cells. In some embodiments, the T cells are able to lyse other suitable types of antigen-expressing targets cells. In some embodiments, the T-lymphocytes in the compositions do not significantly lyse non-cancerous autologous target cells. In some embodiments, the T cells in the compositions do not significantly lyse non-cancerous autologous or allogenic target cells.

### Specific Example of Production of Neoantigen-Specific T Cells

The neoantigen-specific T cells may be produced by contacting a population of antigen presenting cells (APCs) (e.g., dendritic cells (DCs), B cells, monocytes, or a mixture thereof) with an overlapping library of peptides or libraries of peptides (either or which may be referred to as a pepmix). The peptides of a given library encompass a neoantigen (a mutated neoantigen sequence), and in specific embodiments the mutated sequence is located at different positions within each of the individual peptides. This allows for enhanced epitope processing and presentation of the immunogenic epitope peptide as compared to when all of the peptides in the library are substantially identical. In some cases, the APCs are exposed to multiple libraries at substantially the same time, and each of the libraries encompass peptides that span a different neoantigen from another neoantigen. Such contact between the APCs and the pepmix produces pepmix-loaded APCs.

Following this, the pepmix-loaded APCs are exposed to a sufficient amount of peripheral blood mononuclear cells (PBMC) from any individual (including an individual in need of the cell therapy of the method or another individual or individuals that are not in need of the therapy). Such exposure between the two cell populations occurs under suitable conditions in vitro to produce the desired neoantigen-specific T cells, and this step utilizes conditions that stimulate the production of the neoantigen-specific T cells that are capable of responding to at least one neopeptide.

In alternative embodiments, instead of exposing the pepmix to APCs to produce pepmix-loaded APCs and then exposing the pepmix-loaded APCs to PBMCs, one can exclude the APC exposure and instead expose the pepmix library or libraries to the PBMCs directly, given the presence of APCs in the PBMCs (e.g., B cells, monocytes, etc.). In both cases, one or more stimulation steps are utilized to produce neoantigen-specific T cells.

PBMCs may be isolated from an individual in need of treatment or diagnosis or from a healthy donor, including a donor that does not have any cancer or does not have a precancerous condition. The isolation of the PBMCs may occur by any suitable method, and, by way of example, it may be density centrifugation (gradient) (Ficoll-Paque), using cell preparation tubes (CPTs), or using SepMate tubes with freshly collected blood. The PBMC can comprise lymphocytes, monocytes, and dendritic cells. The lymphocytes can include T cells, B cells, and NK cells. In some embodiments, the PBMCs as used herein may be cultured or cryopreserved. In some embodiments, the process of culturing or cryopreserving the cells can include contacting the cells in culture with one or more neoantigens under suitable culture conditions to stimulate and expand neoantigen-specific T cells.

In some embodiments, the process of culturing or cryopreserving the cells can include contacting the cells in culture with one or more neo-epitopes related to one or more neoantigens under suitable culture conditions. In some embodiments, contacting the PBMCs with one or more neoantigens, or one or more neo-epitopes from one or more neoantigens, stimulates and expands a population of neoantigen-specific T cells from the respective donor's MNCs or PMBCs.

Any stimulation step may employ particular medium and particular tissue culture vessels or substrates. The vessels may be flasks or tissue culture-treated or non-tissue culture treated plates, and the vessels may or may not comprise a gas permeable membrane. In some cases, the stimulation occurs in the presence of one or more particular media components, including one or more particular cytokines. In some cases, a specific combination of cytokines is utilized, such as IL-7, IL-12, IL-15 and/or IL-6. There may or may not be a second or more stimulation step, and in such cases the media and/or vessel may or may not be the same as was utilized in the initial stimulation. A second or subsequent stimulation step may utilize a different combination of cytokines compared to a first stimulation step, and one or more cytokines in different stimulations may or may not overlap. In specific cases, a second or subsequent stimulation step utilizes media that comprises IL-7 and one or both of IL-15 and IL-2.

The neoantigen-specific T cell lines may have one or more certain characteristics, including comprising CD4+ T cells and/or CD8+ T cells. In a specific case, the neoantigen-specific T cell lines are predominantly CD8+ and comprise T cells derived from both central (CD62L+) and effector memory (CD62L-) populations. The neoantigen-specific T cells may produce one or more Th1 cytokines, including when stimulated by the neoantigen(s). Examples of the cytokines include IFN-gamma, IL-2, IL-10, and TNF-alpha/beta. In some embodiments, the neoantigen-specific T cells do not significantly lyse non-cancer cells in the recipient individual. The neoantigen-specific T cell lines may or may not be cryopreserved following production.

Once the neoantigen-specific T cells have been stimulated for expansion, they may be characterized or assessed, such as for neoantigen specificity and/or cytotoxicity. In specific embodiments, the neoantigen-specific T cells are assessed for secretion of appropriate effector molecules when stimulated with the appropriate neopeptide(s) but not the corresponding wild-type sequence peptide (or the wild-type peptides stimulates to a lesser degree). In specific cases, the effector cytokines include IL-2, TNF-α, IFNγ, and/or Granzyme B. Assays that may be employed include ELIspot, Flurospot and intracellular cytokine assays, and specifically killed neoantigen-expressing or peptide-pulsed autologous or HLA-matched or partially HLA-matched target cells in traditional Cr⁵¹ release assays.

In general embodiments, a population of neoantigen-specific T-lymphocytes are selected following expansion of a library of T cells produced following exposure to autologous dendritic cells loaded with overlapping peptides that span the targeted neoantigen sequence. The produced cell lines are screened for TCRs that are specific and selective for the neoantigen. The cells may be further expanded and used if appropriate for a recipient individual, or the TCR may be cloned and transfected or transduced into cells of any kind, including immune cells of any kind, when appropriate for a neoantigen and HLA matching.

In some embodiments, the neoantigens to which the cells are directed are from the TP53, AKT1, ESR1, PIK3CA. ERBB2. FRMPD3. GOLGA6L6. HISTIH2AE. MUC4. NBPF12, or SF3B1 genes. In specific cases, the neoantigens are as follows: AKT1 E17K: ESR1 K303R: ESR1 Y537S: ESR1 D538G: PIK3CA E542K: PIK3CA E545K: PIK3CA H1047R; PIK3CA H1047L; TP53 R175H: TP53 R248Q: TP53 R248W: TP53 R273C: TP53 R273H: ERBB2 L755S; ESR1 E380Q: ESR1 L536P: ESR1 S463P: ESR1 Y537C: ESR1 Y537N; EXOC4 S21L; FRMPD3 Q1757E; GOLGA6L6 M472I: HISTIH2AE K128M; MUC4 S2858P; NBPF12 E125Q; PIK3CA E453K: PIK3CA E726K: PIK3CA N345K: PIK3CA Q546K: SF3B1 K700E; TP53 G245S: TP53 H179R: TP53 H193R: TP53 1195T: TP53 R282W; or TP53 Y220C. Thus, the method that produces the neoantigen-specific T cells may produce TCRs that are directed to a neoantigen from the TP53, AKT1, ESR1, PIK3CA. ERBB2. FRMPD3. GOLGA6L6. HISTIH2AE. MUC4. NBPF12, or SF3B1 genes, including for the noted specific neoantigens. The TCRs that are specific for these neoantigens are sequenced and included in one or more immunotherapeutic compositions.

Examples of the variable regions of the specific TCR alpha and beta chains produced by methods of the disclosure are provided below. In specific cases, the nucleic acid sequence of any alpha and/or beta chain may be codon optimized.

TP53 R248W-specific TCR:
Alpha
(SEQ ID NO: 4)
ATGATATCCTTGAGAGTTTTACTGGTGATCCTGTGGCTTCAGTTAAGCTGGGTTTGGA
GCCAACGGAAGGAGGTGGAGCAGGATCCTGGACCCTTCAATGTTCCAGAGGGAGCC
ACTGTCGCTTTCAACTGTACTTACAGCAACAGTGCTTCTCAGTCTTTCTTCTGGTACA
GACAGGATTGCAGGAAAGAACCTAAGTTGCTGATGTCCGTATACTCCAGTGGTAAC
GAAGATGGAAGGTTTACAGCACAGCTCAATAGAGCCAGCCAGTATATTTCCCTGCTC
ATCAGAGACTCCAAGCTCAGTGATTCAGCCACCTACCTCTGTGCGGTCCTCGGGTCA
GGAGGAAGCTACATACCTACATTTGGAAGAGGAACCAGCCTTATTGTTCATCCGTAT
(SEQ ID NO: 12)
MISLRVLLVILWLQLSWVWSQRKEVEQDPGPFNVPEGATVAFNCTYSNSASQSFFWYR
QDCRKEPKLLMSVYSSGNEDGRFTAQLNRASQYISLLIRDSKLSDSATYLCAVLGSGGS
YIPTFGRGTSLIVHPY
Beta
(SEQ ID NO: 5)
ATGGATACCTGGCTCGTATGCTGGGCAATTTTTAGTCTCTTGAAAGCAGGACTCACA
GAACCTGAAGTCACCCAGACTCCCAGCCATCAGGTCACACAGATGGGACAGGAAGT
GATCTTGCGCTGTGTCCCCATCTCTAATCACTTATACTTCTATTGGTACAGACAAATC
TTGGGGCAGAAAGTCGAGTTTCTGGTTTCCTTTTATAATAATGAAATCTCAGAGAAG
TCTGAAATATTCGATGATCAATTCTCAGTTGAAAGGCCTGATGGATCAAATTTCACT
CTGAAGATCCGGTCCACAAAGCTGGAGGACTCAGCCATGTACTTCTGTGCCAGCAGT
GCAGAAGGGGCGCAAGATACGCAGTATTTTGGCCCAGGCACCCGGCTGACAGTGCT
CGAG
(SEQ ID NO: 13)
MDTWLVCWAIFSLLKAGLTEPEVTQTPSHQVTQMGQEVILRCVPISNHLYFYWYRQILG
QKVEFLVSFYNNEISEKSEIFDDQFSVERPDGSNFTLKIRSTKLEDSAMYFCASSAEGAQD
TQYFGPGTRLTVLE
ESR1 Y537S and PIK3CA E545K-specific TCR
Alpha
(SEQ ID NO: 6)
ATGGAAACTCTCCTGGGAGTGTCTTTGGTGATTCTATGGCTTCAACTGGCTAGGGTG
AACAGTCAACAGGGAGAAGAGGATCCTCAGGCCTTGAGCATCCAGGAGGGTGAAA
ATGCCACCATGAACTGCAGTTACAAAACTAGTATAAACAATTTACAGTGGTATAGAC
AAAATTCAGGTAGAGGCCTTGTCCACCTAATTTTAATACGTTCAAATGAAAGAGAGA
AACACAGTGGAAGATTAAGAGTCACGCTTGACACTTCCAAGAAAAGCAGTTCCTTG
TTGATCACGGCTTCCCGGGCAGCAGACACTGCTTCTTACTTCTGTGCTACGCTCGATA
ACTATGGTCAGAATTTTGTCTTTGGTCCCGGAACCAGATTGTCCGTGCTGCCCTAT
(SEQ ID NO: 14)
METLLGVSLVILWLQLARVNSQQGEEDPQALSIQEGENATMNCSYKTSINNLQWYRQN
SGRGLVHLILIRSNEREKHSGRLRVTLDTSKKSSSLLITASRAADTASYFCATLDNYGQN
FVFGPGTRLSVLPY
Beta
(SEQ ID NO: 7)
ATGGGAATCAGGCTCCTCTGTCGTGTGGCCTTTTGTTTCCTGGCTGTAGGCCTCGTAG
ATGTGAAAGTAACCCAGAGCTCGAGATATCTAGTCAAAAGGACGGGAGAGAAAGTT
TTTCTGGAATGTGTCCAGGATATGGACCATGAAAATATGTTCTGGTATCGACAAGAC
CCAGGTCTGGGGCTACGGCTGATCTATTTCTCATATGATGTTAAAATGAAAGAAAAA
GGAGATATTCCTGAGGGGTACAGTGTCTCTAGAGAGAAGAAGGAGCGCTTCTCCCT
GATTCTGGAGTCCGCCAGCACCAACCAGACATCTATGTACCTCTGTGCCAGCAGTCT
AGAACCGATTGGGGATAGTAATGAAAAACTGTTTTTTGGCAGTGGAACCCAGCTCTC
TGTCTTGGAG
(SEQ ID NO: 15)
MGIRLLCRVAFCFLAVGLVDVKVTQSSRYLVKRTGEKVFLECVQDMDHENMFWYRQD
PGLGLRLIYFSYDVKMKEKGDIPEGYSVSREKKERFSLILESASTNQTSMYLCASSLEPIG
DSNEKLFFGSGTQLSVLE
ESR1 Y537S and D538G specific TCR # 1
Alpha
(SEQ ID NO: 8)
ATGTGGGGAGCTTTCCTTCTCTATGTTTCCATGAAGATGGGAGGCACTGCAGGACAA
AGCCTTGAGCAGCCCTCTGAAGTGACAGCTGTGGAAGGAGCCATTGTCCAGATAAA
CTGCATGTACCAGACATCTGGGTTTTATGGGCTGTCCTGGTACCAGCAACATGATGG
CGGAGCACCCACATTTCTTTCTTACAATGCTCTGGATGGTTTGGAGGAGACAGGTCG
TTTTTCTTCATTCCTTAGTCGCTCTGATAGTTATGGTTACCTCCTTCTACAGGAGCTCC
AGATGAAAGACTCTGCCTCTTACTTCTGCGCTGTGAGAGGCGACGATGGTCAGAATT
TTGTCTTTGGTCCCGGAACCAGATTGTCCGTGCTGCCCTAT
(SEQ ID NO: 16)
MWGAFLLYVSMKMGGTAGQSLEQPSEVTAVEGAIVQINCMYQTSGFYGLSWYQQHDG
GAPTFLSYNALDGLEETGRFSSFLSRSDSYGYLLLQELQMKDSASYFCAVRGDDGQNFV
FGPGTRLSVLPY
Beta
(SEQ ID NO: 9)
ATGGGCTGCAGGCTGCTCTGCTGTGCGGTTCTCTGTCTCCTGGGAGCAGTTCCCATA
GACACTGAAGTTACCCAGACACCAAAACACCTGGTCATGGGAATGACAAATAAGAA
GTCTTTGAAATGTGAACAACATATGGGGCACAGGGCTATGTATTGGTACAAGCAGA
AAGCTAAGAAGCCACCGGAGCTCATGTTTGTCTACAGCTATGAGAAACTCTCTATAA
ATGAAAGTGTGCCAAGTCGCTTCTCACCTGAATGCCCCAACAGCTCTCTCTTAAACC
TTCACCTACACGCCCTGCAGCCAGAAGACTCAGCCCTGTATCTCTGCGCCAGCAGCC
AAGGGAGGGGTGGGAATCAGCCCCAGCATTTTGGTGATGGGACTCGACTCTCCATC
CTAGAG
(SEQ ID NO: 17)
MGCRLLCCAVLCLLGAVPIDTEVTQTPKHLVMGMTNKKSLKCEQHMGHRAMYWYKQ
KAKKPPELMFVYSYEKLSINESVPSRFSPECPNSSLLNLHLHALQPEDSALYLCASSQGRGG
NQPQHFGDGTRLSILE
ESR1 Y537S and D538G specific TCR # 2
Alpha
(SEQ ID NO: 10)
ATGCTGACTGCCAGCCTGTTGAGGGCAGTCATAGCCTCCATCTGTGTTGTATCCAGC
ATGGCTCAGAAGGTAACTCAAGCGCAGACTGAAATTTCTGTGGTGGAGAAGGAGGA
TGTGACCTTGGACTGTGTGTATGAAACCCGTGATACTACTTATTACTTATTCTGGTAC
AAGCAACCACCAAGTGGAGAATTGGTTTTCCTTATTCGTCGGAACTCTTTTGATGAG
CAAAATGAAATAAGTGGTCGGTATTCTTGGAACTTCCAGAAATCCACCAGTTCCTTC
AACTTCACCATCACAGCCTCACAAGTCGTGGACTCAGCAGTATACTTCTGTGCTCTG
AGTGAGGCGACCGGTAACCAGTTCTATTTTGGGACAGGGACAAGTTTGACGGTCATT
CCAAAT
(SEQ ID NO: 18)
MLTASLLRAVIASICVVSSMAQKVTQAQTEISVVEKEDVTLDCVYETRDTTYYLFWYKQ
PPSGELVFLIRRNSFDEQNEISGRYSWNFQKSTSSFNFTITASQVVDSAVYFCALSEATGNQ
FYFGTGTSLTVIPN
Beta
(SEQ ID NO: 11)
ATGGGCACCAGGCTCCTCTGCTGGGCGGCCCTCTGTCTCCTGGGAGCAGAACTCACA
GAAGCTGGAGTTGCCCAGTCTCCCAGATATAAGATTATAGAGAAAAGGCAGAGTGT
GGCTTTTTGGTGCAATCCTATATCTGGCCATGCTACCCTTTACTGGTACCAGCAGATC
CTGGGACAGGGCCCAAAGCTTCTGATTCAGTTTCAGAATAACGGTGTAGTGGATGAT
TCACAGTTGCCTAAGGATCGATTTTCTGCAGAGAGGCTCAAAGGAGTAGACTCCACT
CTCAAGATCCAGCCTGCAAAGCTTGAGGACTCGGCCGTGTATCTCTGTGCCAGCAGC
TTAGGTGGGCCGTACAATGAGCAGTTCTTCGGGCCAGGGACACGGCTCACCGTGCTA
GAG
(SEQ ID NO: 19)
MGTRLLCWAALCLLGAELTEAGVAQSPRYKIIEKRQSVAFWCNPISGHATLYWYQQILG
QGPKLLIQFQNNGVVDDSQLPKDRFSAERLKGVDSTLKIQPAKLEDSAVYLCASSLGGP
YNEQFFGPGTRLTVLE

### IV. T Cell Receptors (TCR)

Embodiments of the disclosure encompass engineered T cell receptors (TCRs) (not present in nature) that were produced by the hand of man. In particular embodiments, donor peripheral blood, patient peripheral blood, or both are utilized to activate native tumor-specific T cells using a Th1-polarizing, pro-proliferative cytokine cocktail (e.g., two or more cytokines selected from the group consisting of IL-7, IL-12, IL-15 and IL-6) and autologous dendritic cells loaded with overlapping peptides spanning the targeted neoantigen sequence. The resultant lines were screened for neoantigen specificity and selectivity and also using HLA-blocking antibodies to identify HLA class restriction. The resultant TCRs are sequenced and engineered to generate transgenic neoantigen-specific TCR (Neo-TCR) T cells.

In some cases, to redirect a polyclonal T cell population against one single mutation, one can engineer the T cells to express the mutant-specific TCR. As one example, one can synthesize the identified αand β neoantigen-specific TCR sequences and clone them into vectors of any kind, including viral (retroviral, lentiviral, adenoviral, or adeno-associated viral) or non-viral (plasmid or transposon). In some cases, to avoid mispairing with native TCR sequences in the cells, the human constant region of the transgenic TCRs may be replaced, such as with murine TCR constant regions and to include sequences that stabilize neo-TCR α and β chains to guarantee appropriate transgenic TCR pairing. Once functionality is confirmed (see elsewhere herein), one can refer to the sequencing data to identify signatures associated with TCRs that are linked to high cytotoxic killing.

In specific embodiments, the engineered TCR sequences are particular and may comprise sequence encoded by DNA as follows:
(a) an alpha chain comprising SEQ ID NO:4 or comprising at least 85% identity to SEQ ID NO: 4; and a beta chain comprising SEQ ID NO:5 or comprising at least 85% identity to SEQ ID NO: 5;
(b) an alpha chain comprising SEQ ID NO:6 or comprising at least 85% identity to SEQ ID NO: 6; and a beta chain comprising SEQ ID NO:7 or comprising at least 85% identity to SEQ ID NO: 7:
(c) an alpha chain comprising SEQ ID NO:8 or comprising at least 85% identity to SEQ ID NO: 8; and a beta chain comprising SEQ ID NO:9 or comprising at least 85% identity to SEQ ID NO: 9: or
(d) an alpha chain comprising SEQ ID NO:10 or comprising at least 85% identity to SEQ ID NO: 10; and a beta chain comprising SEQ ID NO: 11 or comprising at least 85% identity to SEQ ID NO: 11.

In specific embodiments, the engineered TCR sequences are particular and may comprise the following sequence:
(a) an alpha chain comprising SEQ ID NO:12 or comprising at least 85% identity to SEQ ID NO: 12; and a beta chain comprising SEQ ID NO: 13 or comprising at least 85% identity to SEQ ID NO: 13:
(b) an alpha chain comprising SEQ ID NO: 14 or comprising at least 85% identity to SEQ ID NO: 14; and a beta chain comprising SEQ ID NO: 15 or comprising at least 85% identity to SEQ ID NO: 15:
(c) an alpha chain comprising SEQ ID NO: 16 or comprising at least 85% identity to SEQ ID NO: 16; and a beta chain comprising SEQ ID NO: 17 or comprising at least 85% identity to SEQ ID NO: 17: or
(d) an alpha chain comprising SEQ ID NO: 18 or comprising at least 85% identity to SEQ ID NO: 18; and a beta chain comprising SEQ ID NO: 19 or comprising at least 85% identity to SEQ ID NO: 19.

Thus, in some embodiments, the cells express recombinant TCRs. The TCR may comprise a variable alpha and beta chain (also known as TCRalpha and TCRbeta, respectively) that is capable of specifically binding to an antigen peptide bound to a MHC receptor. In certain embodiments, the engineered TCR has a variable alpha chain encoded by SEQ ID NO: 4, 6, 8, or 10 and/or a beta chain of SEQ ID NO: 5, 7, 9, or 11. In some embodiments, the TCR has a variable alpha chain that is encoded by sequence that is at least 85, 90, 91, 92, 93, 94, 95, 96, 97, 98, or 99% identical to SEQ ID NO: 4, 6, 8, or 10. In some embodiments, the TCR has a variable beta chain that is at least 85, 90, 91, 92, 93, 94, 95, 96, 97, 98, or 99% identical to SEQ ID NO: 5, 7, 9, or 11, respectively. In certain embodiments, the engineered TCR has a variable alpha chain comprising SEQ ID NO: 12, 14, 16, or 18 and/or a beta chain of SEQ ID NO: 13, 15, 17, or 19. In some embodiments, the TCR has a variable alpha chain that comprises sequence that is at least 85, 90, 91, 92, 93, 94, 95, 96, 97, 98, or 99% identical to SEQ ID NO: 12, 14, 16, or 18. In some embodiments, the TCR has a variable beta chain that is at least 85, 90, 91, 92, 93, 94, 95, 96, 97, 98, or 99% identical to SEQ ID NO: 13, 15, 17, or 19, respectively.

Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, Immunobiology: The Immune System in Health and Disease, 3^{rd} Ed., Current Biology Publications, p. 433, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable alpha chain and variable beta chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., PNAS U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the beta-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Va or Vp: typically amino acids 1 to 116 based on Kabat numbering Kabat et al., “Sequences of Proteins of Immunological Interest, US Dept. Health and Human Services, Public Health Service National Institutes of Health, 1991, 5.sup.th ed.) at the N-terminus, and one constant domain (e.g., α-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the alpha and beta chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (gamma, delta, and epsilon) in mammals and the zeta-chain. For example, in mammals the complex can contain a CD3gamma chain, a CD3delta chain, two CD3epsilon chains, and a homodimer of CD3zeta chains. The CD3gamma, CD3delta, and CD3epsilon chains are highly related cell surface proteins of the immunoglobulin superfamily containing a single immunoglobulin domain. The transmembrane regions of the CD3gamma, CD3delta, and CD3epsilon chains are negatively charged, which is a characteristic that allows these chains to associate with the positively charged T cell receptor chains. The intracellular tails of the CD3gamma, CD3delta, and CD3epsilon chains each contain a single conserved motif known as an immunoreceptor tyrosine-based activation motif or ITAM, whereas each CD3zeta chain has three. Generally, ITAMs are involved in the signaling capacity of the TCR complex. These accessory molecules have negatively charged transmembrane regions and play a role in propagating the signal from the TCR into the cell. The CD3zeta chains, together with the TCR, form what is known as the T cell receptor complex.

In some embodiments, the TCR may be a heterodimer of two chains alpha and beta (or optionally gamma and delta) or it may be a single chain TCR construct. In some embodiments, the TCR is a heterodimer containing two separate chains (alpha. and beta chains or gamma and delta chains) that are linked, such as by a disulfide bond or disulfide bonds. In some embodiments, a TCR for a target antigen (e.g., a cancer neoantigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell). In some embodiments, the T cells can be obtained from in vivo isolated cells. In some embodiments, a high-affinity T cell clone can be isolated from a patient, and the TCR isolated. In some embodiments, the T-cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808). In some embodiments, phage display is used to isolate TCRs against a target antigen (see, e.g., Varela-Rohena et al. (2008) Nat Med. 14:1390-1395 and Li (2005) Nat Biotechnol. 23:349-354). In some embodiments, the TCR or antigen-binding portion thereof can be synthetically generated from knowledge of the sequence of the TCR.

### V. Identification of HLA Restriction

In particular embodiments of the disclosure, therapeutic cells are provided to an individual in need thereof when the cancer of the individual has cells that express the neoantigen and when there is HLA matching between the neoantigen and the individual that is the recipient for the cells.

In specific embodiments, Neo-TCR+ T cells are scored for the HLA allele restriction of each Neo-TCR. One can clone each donor HLA allele (A, B and C-class I; and DR, DQ and DP—class II) into transfection or transduction plasmids that are expressed in cells that lack human HLA but that either express the cognate neoantigen or are pulsed with cognate neo-peptide. Only those co-expressing the mutation and relevant HLA will present the neoantigen and induce the reactivity of the Neo-TCR+ T cells (e.g., as measured by IFNγ ELISA or ELISpot analysis, as examples).

### VI. Engineering of Cells

In some embodiments, the neoantigen-specific T cells produced by methods encompassed herein are utilized themselves as compositions for treatment for an individual in need thereof. In other cases, the neoantigen-specific T cells produced by methods encompassed herein are not themselves utilized as a therapeutic composition, but sequences of the TCRs of these produced cells are identified and further utilized in therapeutic compositions, such as other cells. In some embodiments, the TCRs of the neoantigen-specific T cells are analyzed for their sequence, and the sequence of the TCRs is utilized in subsequently engineered T cells having these TCR (or similar, such as greater than 75, 80, 85, 90, or 95% identity) sequences. In specific cases, neoantigen-specific T cells known or demonstrated to be responsive to neopeptides, and not their corresponding wild-type peptides, will have their respective TCR sequences obtained. T cells then are engineered to express the neoantigen-specific TCR to produced engineered T cells (that may also be referred to as engineered neoantigen-specific T cells). The identified αand β TCR sequences may be cloned into an appropriate vector (viral or non-viral) and transduced or transfected into T or other immune (e.g., NK/NKT) cells. Surface expression may be confirmed, such as by flow cytometry. Specificity may be confirmed, such as by cytokine (IL-2, TNF-α, IFNγ, and/or Granzyme) release upon exposure to the neopeptide but not the corresponding wild-type sequence. Cytotoxicity may be confirmed, such as by killing of neopeptide-pulsed targets in a Cr⁵¹ release assay.

For the engineered T cells expressing the neoantigen-specific TCR, measures may be taken to avoid mispairing with native TCR sequences in the T cells being engineered. For example, the constant regions for the engineered TCRs may be non-human, including murine. In addition, or alternatively, one may include sequences in the TCR that stabilize neo-TCR α and β chains to guarantee appropriate transgenic TCR pairing. Examples include the addition of disulphide bonds, hydrophobic modifications, swapping constant domains of αand β chain, disrupting endogenous TCR expression (e.g. via gene knockouts, siRNA, CRISPR, etc.), use of γδ constant domains, incorporation of CD3zeta to TCR, use of single chain TCR format, removal of N-glycosylation sites, specific insertion of the TCR into the TCR locus of the target cells, use of TCR deleted cells and others.

In any cells encompassed herein, including the neoantigen-specific T cells produced by methods encompassed herein and that are used for therapy, or cells that are engineered to express the TCRs of the neoantigen-specific cells produced by methods encompassed herein, may be modified by the hand of man to have one or more modifications. The modifications may be useful to increase the therapeutic efficacy of the cells, to increase the persistence of the cells, or both. The cells may be modified to express one or more heterologous molecules that are not endogenous to the cells, such as one or more synthetic receptors, such as chimeric antigen receptors, TCRs, chimeric cytokine receptors, cytokine receptors, and so forth. In some cases, the cells are modified to express one or cytokines. Such modifications may be modifications such that the non-endogenous molecule(s) is integrated into the cell genome (and this integration may or may not be directed to a specific locus), or the non-endogenous molecule(s) may be present on a vector (viral or non-viral) in the cell.

In particular embodiments, any cells are modified to express the engineered neoantigen-specific TCR, including polyclonal expanded tumor associated antigen-specific T cells. Examples of tumor associated antigens include survivin, MAGE-A4, SSX2, PRAME, and NY-ESO-1.

In some embodiments as an alternative to, or in addition to, the cells expressing one or more non-endogenous molecules, the cells may have disruption of one or more endogenous genes, such as endogenous TCR or HLA molecules. The disrupted gene(s) may be of any kind and the disruption may be produced by any method, including CRISPR, for example. In some cases, the gene that is disrupted is the endogenous TCR of the T cell.

### VII. Methods of Treatment or Prevention

Embodiments of the disclosure encompass methods of treatment or prevention in which a therapeutically effective amount of cells produced by methods encompassed herein are administered to an individual in need thereof. In particular embodiments, there are methods of treating or preventing cancer or reducing the risk of cancer in an individual comprising the step of administering by any suitable route an effective amount of engineered neoantigen-specific T cells or other immunotherapeutic compositions to an individual that has cancer or has a pre-cancerous condition or is at risk for cancer. The treatment methods will treat cancer in which the cancer has one or more neoantigens to which the neoantigen-specific T cells are directed. The prevention methods will prevent cancer in individuals at high risk of developing disease. In some cases, the methods of treatment or prevention allow for killing (including by lysing) of target cells that comprise the neoantigen(s) by contacting the target cells with an effective amount of the neoantigen-specific T cells, and in specific embodiments the contacting occurs in vivo in an individual that is the recipient of the cells.

In some cases, a therapeutic composition that comprises neoantigen-specific T cells are effective against more than one neoantigen because the population of neoantigen-specific T cells in the composition are specifically generated to recognize collectively the more than one antigen. In other cases, a therapeutic composition that comprises neoantigen-specific T cells are effective against more than one neoantigen because two or more independently generated cell lines that each are directed towards different neoantigens have been mixed or combined in the therapeutic composition. In alternative cases, the two or more independently generated cell lines are used in separate formulations for the same individual and may or may not be given at substantially the same time.

In specific embodiments, the individual is known to have cancer, is suspected of having cancer, or is at a higher risk for cancer when compared to the general population (a smoker, has a personal or family history, has exposure to the sun and/or environmental carcinogens, has a cancer-associated virus, such as HPV, is obese, is older, such as older than 40, etc.). Treatment with the produced cells may ameliorate one or more symptoms of cancer, may prevent cancer, may reduce the severity of one or more symptoms, may delay onset of the cancer, or may delay or reduce the extent of metastasis of the cancer.

In certain embodiments of the present disclosure, neoantigen-specific T cells are delivered to an individual in need thereof, such as an individual that has cancer. The neoantigen-specific T cells may be autologous with respect to the individual that has cancer, or the neoantigen-specific T cells may be allogeneic with respect to the individual that has cancer. In allogeneic embodiments, the neoantigen-specific T cells may be at least partially HLA matched as when utilized in an off-the-shelf approach. The cells then mediate direct anti-tumor effects and may also enhance the individual's immune system to attack the cancer cells. In particular embodiments, the treatment methods treat cancers in which at least one neoantigen is associated. In specific embodiments, the individual has cancer harboring one or more of the neoantigens for which the cells are targeted. In specific embodiments, the cancer is lung, breast, colon, hematological, bone, liver, kidney, brain, pancreas, uterine, skin, head and neck, ovarian, endometrial, testicular, stomach, gall bladder, spleen cancer, and so forth. Types of hematological cancers include leukemia (acute lymphocytic leukemia: acute myelogenous leukemia: chronic lymphocytic leukemia: chronic myeloid leukemia, etc.), lymphoma (Hodgkin lymphoma: non-Hodgkin lymphoma: chronic lymphocytic leukaemia (CLL): small lymphocytic lymphoma: etc.) and multiple myeloma (hyperdiploid or non-hyperdiploid). In specific cases, the cancer is primary, minimal residual disease, early cancer, advanced cancer, metastatic cancer, and/or relapsed refractory cancer, for example. The cancer may be of any stage, including stage I, II, III, or IV, for example.

The individual may utilize the treatment method of the disclosure as an initial treatment or after (and/or with) another treatment (e.g., chemotherapy, radiation, surgery, hormone therapy, and/or other types of immunotherapy). The immunotherapy methods may be tailored to the need of an individual with cancer based on the neoantigen, type, and/or stage of cancer, and in at least some cases the immunotherapy may be modified during the course of treatment for the individual. For example, if the cancer metastasizes, then the immunotherapy may change for a neoantigen associated with the metastasis or metastases. In other cases, the immunotherapy may be modified over the course of treatment for another neoantigen present in cancer cells of the individual.

In some embodiments, the present disclosure provides methods for immunotherapy comprising administering a therapeutically effective amount of the immunotherapeutic compositions generated from the TCRs of the neoantigen-specific T cells produced by methods of the present disclosure. In one embodiment, a medical disease or disorder is treated by transfer of cell populations produced by methods herein and that elicit an immune response. In certain embodiments of the present disclosure, cancer is treated by transfer of a cell population produced by methods of the disclosure and that elicits an immune response. Provided herein are methods for treating or delaying progression of cancer in an individual comprising administering to the individual an effective amount of engineered neoantigen-specific cells comprising the desired TCR. The therapeutically effective amount of the produced cells for use in adoptive cell therapy is that amount that achieves a desired effect in a subject being treated. For instance, this can be the amount of neoantigen-specific T cells necessary to inhibit advancement or to cause regression of cancer.

In some cases, the individual is provided with one or more doses of the immunotherapeutic composition, such as engineered immune cells. In some embodiments, the composition as described herein is administered to the subject a plurality of times. In some embodiments, the composition as described herein is administered to the subject more than one time. In some embodiments, the composition as described herein is administered to the subject more than two times. In some embodiments, the composition as described herein is administered to the subject more than three times. In some embodiments, the composition as described herein is administered to the subject more than four times. In some embodiments, the composition as described herein is administered to the subject more than five times. In some embodiments, the composition as described herein is administered to the subject more than six times. In some embodiments, the composition as described herein is administered to the subject more than seven times. In some embodiments, the composition as described herein is administered to the subject more than eight times. In some embodiments, the composition as described herein is administered to the subject more than nine times. In some embodiments, the composition as described herein is administered to the subject more than ten times. In some embodiments, the composition as described herein is administered to the subject a number of times that are suitable for the subjects. In specific cases where the individual is provided with two or more doses of the immune cells, the duration between the administrations should be sufficient to allow time for propagation in the individual, and in specific embodiments the duration between doses is 1, 2, 3, 4, 5, 6, 7, or more days: or 1, 2, 3, 4, or more weeks: or 1, 2, 3, 4, 5, 6, 7, 8, 9, 10, 11, 12 or more months.

In specific embodiments, the cells are provided to an individual in a therapeutically effective amount (in a range from 10³ to 10¹⁰) that ameliorates at least one symptom related to neoantigen-expressing cancer cells in the individual. A therapeutically effective amount may be from 10³ to 10¹⁰, 10³ to 10⁹, 10³ to 10⁸, 10³ to 10⁷, 10³ to 10⁶, 10³ to 10⁵, 10³ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10⁸ to 10¹⁰, 10⁸ to 10⁹, or 10⁹ to 10¹⁰ cells. Thus, in particular embodiments an individual having a particular neoantigen-positive cancer is provided once or multiple times a therapeutically effective amount of cells expressing T cells directed towards the neoantigen.

The produced cell population or other immunotherapeutic composition can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of cells will be dependent on the subject being treated, the severity and type of the affliction, and the manner of administration. In some embodiments, doses that could be used in the treatment of human subjects range from at least 1×10³, at least 1×10⁴, at least 1×10⁵, at least 1×10⁶, at least 1×10⁷, at least 1×10⁸, at least 1×10⁹, or at least 1×10¹⁰ T cells/m². In a certain embodiment, the dose used in the treatment of human subjects ranges from about 1×10⁹ to about 1×10¹⁰ T cells/m². In additional embodiments, a therapeutically effective amount of T cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of T cells is readily determined by one of skill in the art based on the age, weight, sex, and physiological condition of the subject. Effective doses can be extrapolated from dose-response curves derived from in vitro or animal model test systems.

The present disclosure provides methods of treating or preventing cancer comprising administering to a subject in need thereof the compositions or the pharmaceutical compositions as described herein. In some embodiments, the T-lymphocytes between 1×10³ and 1×10⁹ T-lymphocytes/m², 1×10⁴ and 1×10⁸ T-lymphocytes/m², 1×10⁵ and 1×10⁷ T-lymphocytes/m², 1×10⁴ and 1×10⁸ T-lymphocytes/m², 1×10⁶ and 1×10⁹ T-lymphocytes/m², inclusive of all ranges and subranges there between. In some embodiments, the T-lymphocytes are administered to the subject. In some embodiments, the subject is immunocompromised.

Therapeutically effective amounts of the produced cells can be administered by a number of routes, including parenteral administration, for example, intravenous, intraperitoneal, intramuscular, intrasternal, intratumoral, intrathecal, intraventricular, through a reservoir, intraarticular injection, or infusion.

The present disclosure provides methods of lysing a target neoantigen-expressing cancer cell comprising contacting the target cell with the compositions or pharmaceutical compositions as described herein. In some embodiments, the contacting between the target neoantigen-expressing cancer cell and the compositions or pharmaceutical compositions occurs in vivo in a subject. In some embodiments, the contacting between the target cell and the compositions or pharmaceutical compositions occurs in vivo via administration of the neoantigen-specific T-cells to a subject. In some embodiments, the subject is a human.

The present disclosure provides pharmaceutical compositions comprising a polyclonal population of T-cells that recognize a plurality of neoantigens. In some embodiments, the present disclosure provides a polyclonal population of T-cells that recognize a plurality of neoantigens comprising at least one antigen from each of one or more genes.

In some embodiments, the present disclosure provides pharmaceutical compositions comprising the compositions as described herein formulated for intravenous delivery. In some embodiments, the composition as described herein is negative for bacteria. In some embodiments, the composition as described herein is negative for fungi. In some embodiments, the composition as described herein is negative for bacteria or fungi for at least 1 days, at least 2 days, at least 3 days, at least 4 days, at least 5 days, at least 6 days, at least 7 days, at least 8 days, at least 9 days, at least 10 days, in culture. In some embodiments, the composition as described herein is negative for bacteria or fungi for at least 7 days in culture.

The cells of the disclosure may be encompassed in a pharmaceutically acceptable carrier. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

In some embodiments, the pharmaceutical compositions formulated for intravenous delivery exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the pharmaceutical compositions formulated for intravenous delivery are negative for mycoplasma.

Viii. Methods of Diagnosis of Pre-Cancerous or Cancer Expressing Neoantigens

Embodiments of the disclosure include methods of diagnosing a cancer, wherein the cancer is associated with one or more neoantigens (e.g., a sample from an individual suspected of having a cancer has cells that express the neoantigen(s)). Other methods include measuring, assaying, detecting, or analyzing in a sample for one or more particular conditions associated cancer. In specific embodiments, the disclosure includes methods of treatment that are determined, or the need is determined, following methods of measuring, assaying, detecting, or analyzing in a sample for one or more particular conditions associated with a cancer.

Endogenous T cells in individuals with neoantigen-specificity can recognize neoantigens in pre-cancerous and cancerous lesions and develop into memory cells. These neoantigen-specific memory cells may be present in peripheral blood, tumor, lymph nodes, spleen, etc., and their detection can indicate the presence of the cancerous or pre-cancerous lesion in the individual, including for diagnosis. Thus, in specific embodiments, there are methods of measuring for the presence of neoantigen-specific memory cells in a sample from an individual suspected of having or known to have cancer. In some cases, there are methods of detecting neoantigen-specific memory cells in an individual suspected of having cancer. In some cases, there are methods of analyzing or assaying a sample for the presence of neoantigen-specific memory cells in a sample from an individual suspected of having cancer. In specific embodiments, upon identification of neoantigen-specific memory cells in a sample from an individual suspected of having cancer, the individual is appropriately treated with any suitable kind of treatment. The treatment may include engineered neoantigen-specific T cells and/or one or more other cancer treatments, such as chemotherapy, immunotherapy, hormone therapy, radiation, surgery, etc.

The disclosure includes methods of detecting neoantigen-specific memory cells directed against one or more neoantigens in an individual suspected of having or known to cancer, comprising performing a step for detecting the neoantigen-specific memory cells in a sample from the individual and, upon detection, treating the individual with a therapy for the cancer. Also disclosed herein are methods for treating cancer, the method comprising detecting neoantigen-specific T cells in a sample from the individual and administering to the individual an effective amount of neoantigen-specific T cells and/or one or more other cancer treatments, such as chemotherapy, immunotherapy, hormone therapy, radiation, surgery, etc. Methods of the disclosure include methods of treating cancer or preventing cancer in an individual, comprising the step of administering to the individual an effective amount of neoantigen-specific T cells and/or one or more other cancer treatments, such as chemotherapy, immunotherapy, hormone therapy, radiation, surgery, after determining that the individual has neoantigen-specific memory T cells in a sample from the individual.

Thus, embodiments of the disclosure encompass methods of diagnosing cancer by identifying neoantigen specificity in memory cells. The memory cells can be assessed in the absence of an enrichment step in which the memory cells are first contacted with APCs that have been contacted with one or a plurality of pepmix libraries that encompass the neoantigen(s). In other cases, the memory cells can be assessed following an enrichment step in which the memory cells are stimulated with APCs that have been contacted with one or a plurality of pepmix libraries that encompass the neoantigen(s).

The memory T cells (whether or not they have undergone expansion) may be assayed for specificity. In some cases, the memory T cells may be exposed to at least one pepmix library that encompasses one or more neoantigens. Specificity of the memory T cells may be determined by secretion of one or more appropriate effector molecules when stimulated with the appropriate neopeptide(s), such as IL-2, TNF-α, IFNγ, and/or Granzyme B. Assays that may be employed include ELIspot, Flurospot and intracellular cytokine assays, and specifically killed peptide-pulsed autologous or HLA-matched neoantigen-expressing target cells in traditional Cr⁵¹ release assays. Specificities can also be determined by using pentamers or identification of TCR sequences with known reactivity to neopeptides.

In some embodiments of the present methods, the methods further comprise obtaining the sample from the individual. The sample may or may not be stored prior to the present methods steps.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Targeting Common Somatic Mutations in Breast Cancer with Neo-Antigen Specific Adoptive T Cell Therapy

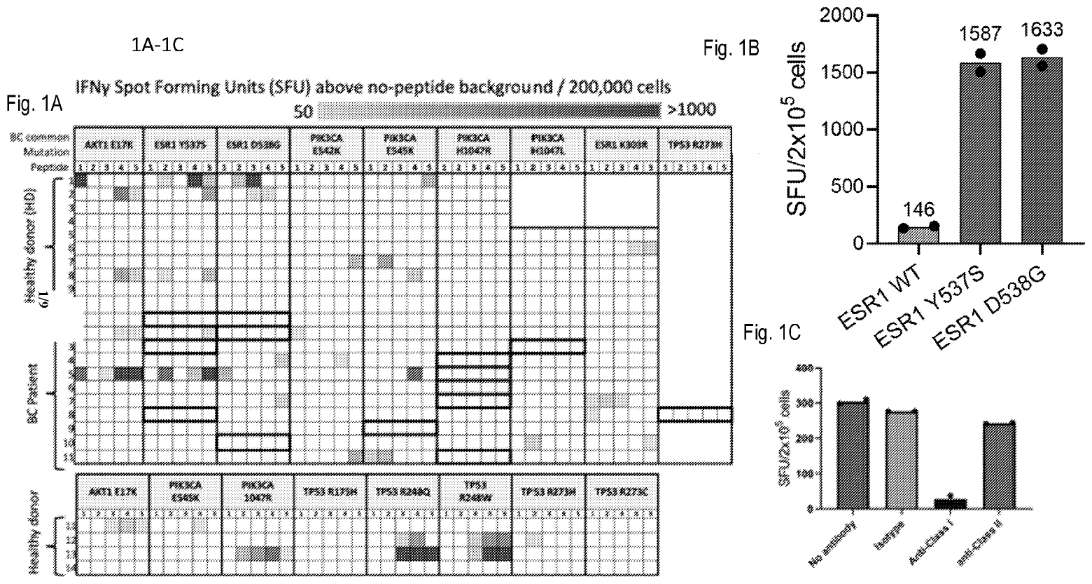

The present example concerns production and use of neo-antigen specific T cell therapy that targets somatic mutations in tumors, including somatic mutations that are unique to an individual's tumor. In initial studies, the inventors have successfully expanded T cells from both healthy donors and breast cancer patients that selectively target common breast cancer neoantigens, demonstrating the feasibility of the approach. Unexpectedly, it was found that neoantigen-reactive cells from cancer patients did not react against the patient's own neo-antigens, consistently failing to recognize the mutations harbored by the patient's own tumor cells (FIG. 1). Although in at least some cases this phenomenon impedes the use of these cells as an autologous therapy, patient's T cells readily recognized other known common neoantigens. In specific embodiments, one can isolate/clone the neoantigen-specific T cell receptors (TCRs) from T cells to generate a repertoire of transgenic TCR vectors^{6,7} that can be used to genetically redirect the patients' T cells against their own tumor mutations.

In particular embodiments, one can establish an expanded library of neoantigen-specific T cells with high specificity and cytotoxicity. Fifteen of 25 T cell lines demonstrate neoantigen specificity for one or more of thirteen-targeted somatic mutations in four genes (TP53, PIK3CA, ESR1, AKT). In particular embodiments of the disclosure, for a patient to benefit from Neo-T cell therapy, their tumor must harbor the mutation recognized by the TCR and must have the specific HLA polymorphism that presents the peptide to which the TCR binds. One can ensure the racial and ethnic diversity of the patients that can benefit from Neo-T cell therapy by isolating TCRs that target common neoantigens and are restricted to HLA alleles present in a broadly representative population.

In specific embodiments, one can expand the library of Neo-T and identify their HLA class restriction. Neo-T may be generated from healthy donor and patient peripheral blood using an established protocol^{8,9} where one activates native tumor-specific T cells using a Th 1-polarizing, pro-proliferative cytokine cocktail and autologous dendritic cells loaded with overlapping peptides spanning the targeted mutation sequence. One can screen Neo-T lines, selecting those that show neoantigen specificity by IFNγ ELIspot assay. FIG. 1A shows results from the first 25 donors screened. Reactive lines may undergo further ELIspot testing to confirm recognition of the mutation but not the wild-type peptide, with HLA blocking antibodies used to identify HLA class restriction (FIGS. 1B-1C).

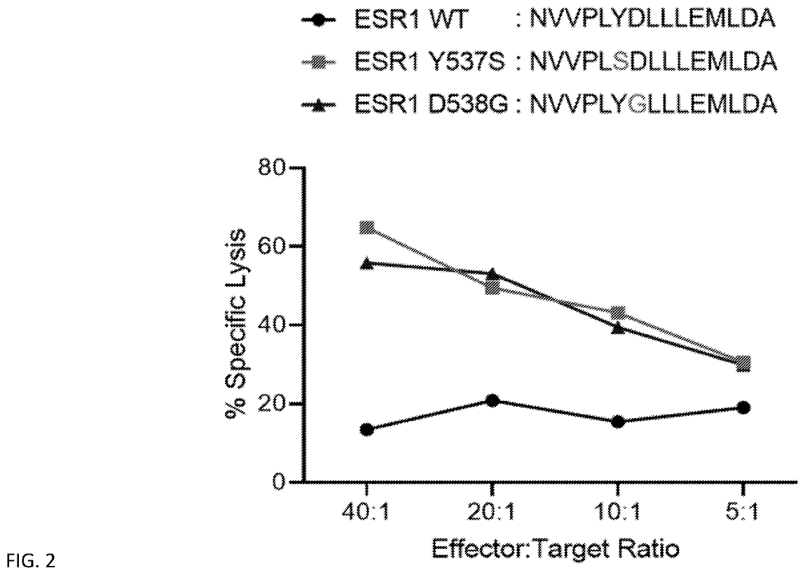

One can measure the polyfunctionality and anti-tumor activity of Neo-T. The neoantigen-reactive T cells may be characterized, such as confirming secretion of one or more effector cytokines (including IL-2 and/or TNF-α, for example) using intracellular staining and ELISA. Their cytotoxic activity can be measured against autologous neoantigen-loaded PHA blasts in a 6-hour Cr⁵¹ release assay (FIG. 2). With these results, one can select the Neo-T lines that recognize and kill breast cancer neoantigen targets with the objective of isolating their neoantigen-specific TCRs (Neo-TCRs).

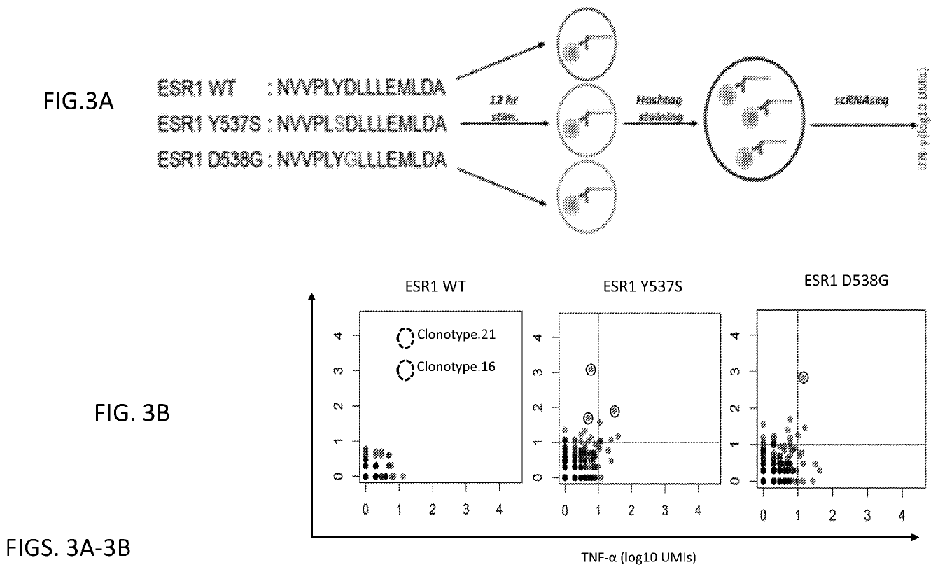

In particular embodiments, one can generate a library of transgenic Neo-TCR vectors (e.g., retroviral vectors) to use as autologous therapy for breast cancer patients. In some cases, one can identify Neo-specific TCRs using single cell RNA sequencing (scRNAseq). The scRNAseq method may be used to identify the TCR sequences of Neo-T¹⁰. Briefly, starting with the 15 reactive lines and extending based on studies referred to above, one can pulse reactive lines with their stimulating neo or corresponding wild-type peptides, labeling each condition with a Hashtag oligo (FIG. 3A). After 3-12 hours, cells are pooled and sent for scRNAseq. Cells that secrete IFNγ and TNFα in response to the mutated but not wild-type peptide are identified and their respective TCR sequences obtained (FIG. 3B). One can also identify Neo-specific TCRs from neoantigen-reactive T cell lines through a sequential process consisting of INFγ-capture, limiting dilution cloning, and 5′ rapid amplification of cDNA ends (5′ RACE). Briefly, one can pulse reactive lines with their stimulating neo peptides for 3-6 hrs, after which a process called IFNγ-capture is performed that captures IFNγ onto the surface of IFNγ-secreting T cells, allowing for these cells to be detected with and anti-IFNγ antibody and isolated by magnetic bead separation. After isolation, limiting dilution expansion is performed, in which single cells are added to wells of a 96-well plate and expanded in a special T cell expansion medium (e.g., including irradiated allogeneic PBMC feeder cells, IL2, PHA, and/or anti-CD3 antibody). After about 2 weeks, expanding T cell clones can be rescreened for neoantigen specificity, and further expanded if necessary. RNA is then extracted from neoantigen-specific T cell clonal lines, and partial TCR alpha and beta chains are amplified by 5′ RACE, and amplicons are subcloned and submitted for Sanger sequencing. Six Neo-TCRs were isolated from the first 3 Neo-T cell lines selected (Healthy Donors 1, 8 and 13 (Donor 1 TCRs were obtained via scRNAseq, and the Donor 8 and 13 lines were obtained via the IFNγ capture method)). These TCRs allow redirection of a patient's immune system against their tumor.

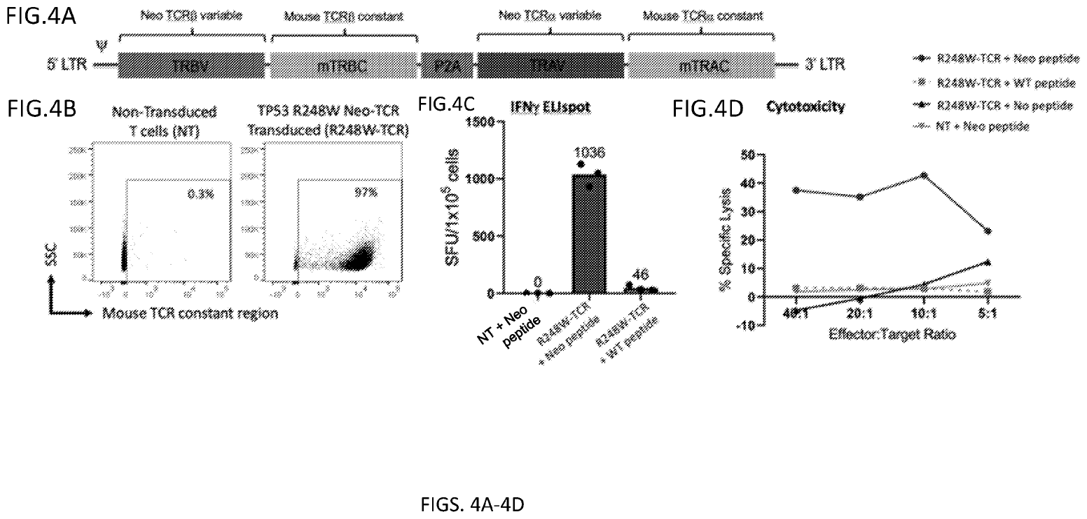

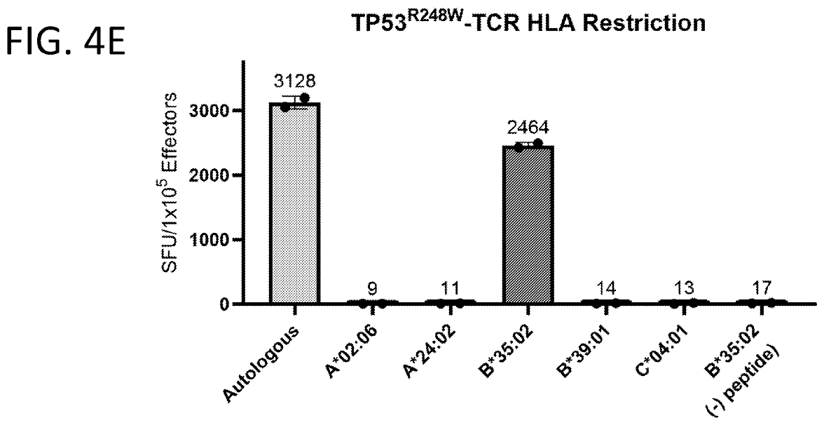

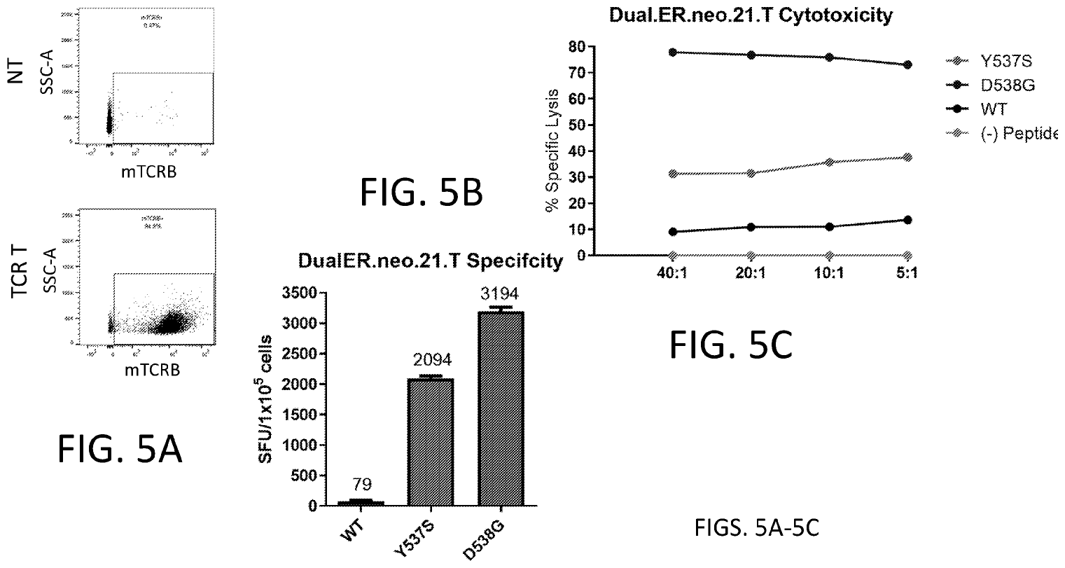

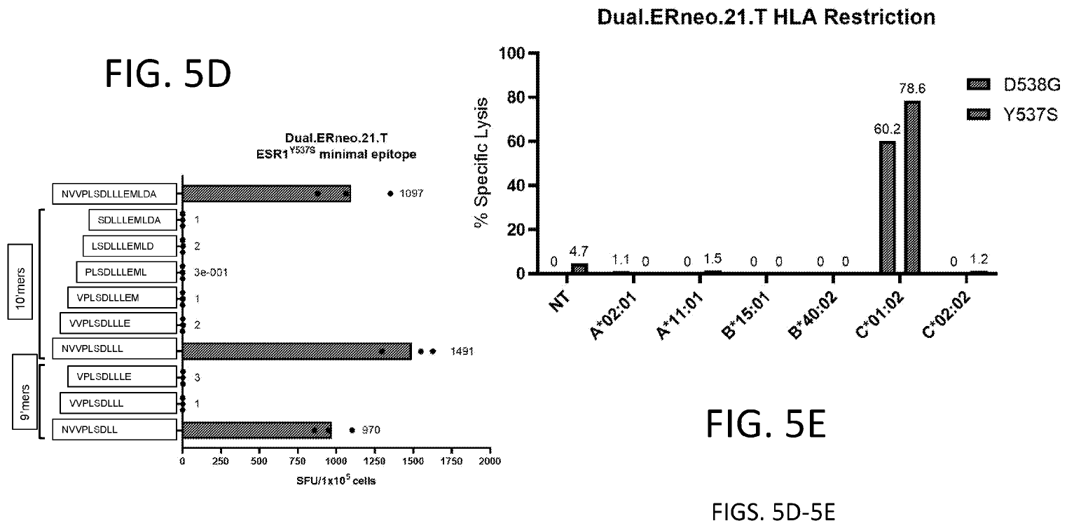

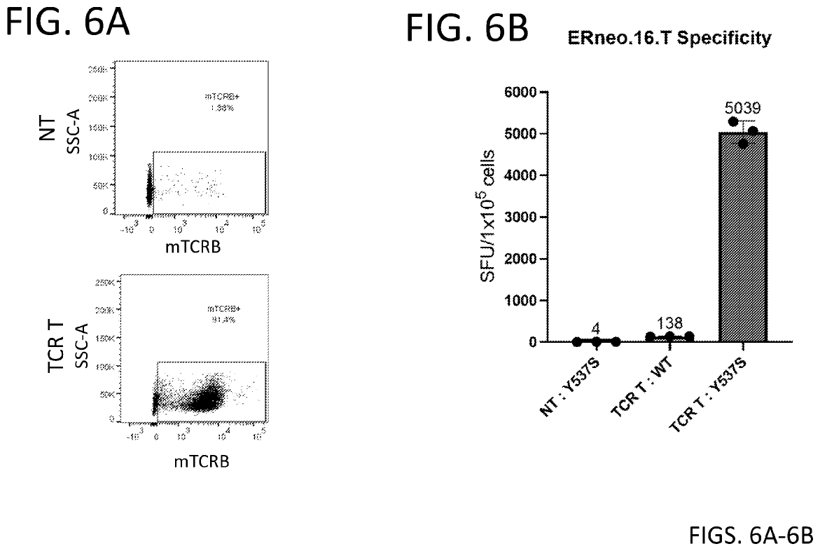

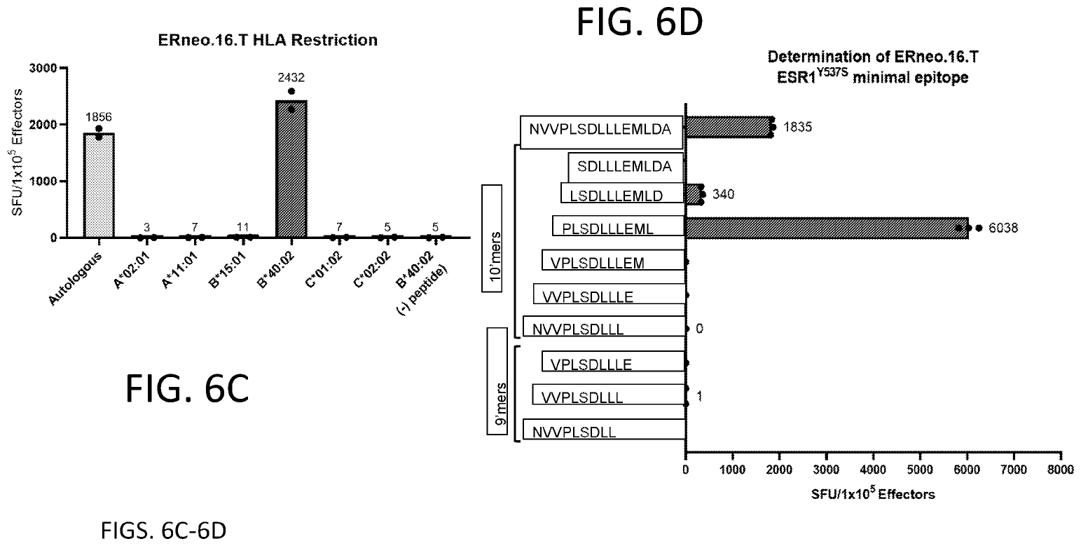

Transgenic neoantigen-specific TCR (Neo-TCR) T cells may be generated. To redirect a polyclonal T cell population against one single mutation, one can engineer them to express the mutant-specific TCR. One can synthesize identified αand β Neo-TCR sequences and clone them into retroviral vectors. To avoid mispairing with native TCR sequences, the human constant region of the transgenic TCRs may be replaced with murine TCR constant regions and one can include sequences that stabilize neo-TCR α and β chains to generate appropriate transgenic TCR pairing. One can then transduce OKT3/CD28 blasts with the Neo-TCR transgene and confirm surface expression by flow cytometry. INFγ release in response to the neo-peptide (but not wild type) and killing of neopeptide pulsed targets in a Cr⁵² release assay (FIGS. 4, 5, and 6). In order to improve the ability to choose for future lines, the RNAseq data may be referred to for signatures associated to the TCRs with high cytotoxic killing.

Identification of HLA restriction. A patient population may be established that will benefit from the Neo-TCR+ T cells by identifying the HLA allele restriction of each Neo-TCR. One can clone each donor HLA allele [A, B and C-class I: DR, DQ and DP-class II] into transfection plasmids, which will be individually expressed in COS-7 cells (monkey cell line lacking human HLA) along with the mutation gene sequence. Only those co-expressing the mutation and relevant HLA will induce Neo-TCR+ T cell reactivity, as measured by IFNγ ELISA^{11,12}.

Since 15 of the already generated lines have demonstrated specificity for the target neoantigens, one can expand the current library of TP53/PIK3CA/ESR1/AKT Neo-T lines. The present disclosure indicates the feasibility of obtaining and cloning Neo-TCRs into vectors, such as retroviral vectors. In particular embodiments, transgenic Neo-TCR+ T cells will kill mutation-expressing autologous and HLA-matched cells. The scRNAseq results facilitate the preparation of a transgenic TCR library to redirect polyclonal T cells to patient-specific mutations.

In particular embodiments, there is establishment of the immunogenicity of TP53, ESR1, PIK3CA and AKT mutations and the ability to generate transgenic neoantigen-specific TCRs for the treatment of patients with breast cancer.

### REFERENCES

The following references, to the extent that they provide exemplary procedural or other details supplementary to those set forth herein, are specifically incorporated herein by reference.
1. Yamamoto T N, Kishton R J, Restifo N P. Developing neoantigen-targeted T cell-based treatments for solid tumors. Nat Med. October 2019: 25 (10): 1488-1499. doi: 10.1038/s41591-019-0596-y
2. Zacharakis N, Chinnasamy H, Black M, et al. Immune recognition of somatic mutations leading to complete durable regression in metastatic breast cancer. Nat Med. June 2018: 24 (6): 724-730. doi: 10.1038/s41591-018-0040-8
3. COSMIC catalog of somatic mutations.

https://cancer.sanger.ac.uk/cosmic/browse/tissue?wgs=off&sn=breast&ss=all&hn=carcinoma&s
h=all&in=t&src=tissue&all_data=n

4. Fuqua S A, Rechoum Y, Gu G. ESR1 Mutations in Cell-Free DNA of Breast Cancer: Predictive “Tip of the Iceberg”. JAMA Oncol. Oct] 2016: 2 (10): 1315-1316. doi: 10.1001/jamaoncol.2016.1268
5. Li S, Shen D, Shao J, et al. Endocrine-therapy-resistant ESR1 variants revealed by genomic characterization of breast-cancer-derived xenografts. Cell Rep. Sep. 26 2013: 4 (6): 1116-30. doi: 10.1016/j.celrep.2013.08.022
6. Zhao Q, Jiang Y, Xiang S, et al. Engineered TCR-T Cell Immunotherapy in Anticancer

Precision Medicine: Pros and Cons. Front Immunol. 2021:12:658753. doi: 10.3389/fimmu.2021.658753
7. Nagarsheth N B, Norberg S M, Sinkoe A L, et al. TCR-engineered T cells targeting E7 for patients with metastatic HPV-associated epithelial cancers. Nat Med. March 2021: 27 (3): 419-425. doi: 10.1038/s41591-020-01225-1
8. Vasileiou S, Turney A M, Kuvalekar M, et al. Rapid generation of multivirus-specific T lymphocytes for the prevention and treatment of respiratory viral infections. Haematologica. January 2020; 105 (1): 235-243. doi: 10.3324/haematol.2018.206896
9. Gerdemann U, Katari U, Christin A S, et al. Cytotoxic T lymphocytes simultaneously targeting multiple tumor-associated antigens to treat EBV negative lymphoma. Mol Ther. December 2011; 19 (12): 2258-68. doi: 10.1038/mt.2011.167
10. Lu Y C, Zheng Z, Lowery F J, et al. Direct identification of neoantigen-specific TCRs from tumor specimens by high-throughput single-cell sequencing. J Immunother Cancer. July 2021; 9 (7) doi: 10.1136/jitc-2021-002595
11. Cafri G, Yossef R, Pasetto A, et al. Memory T cells targeting oncogenic mutations detected in peripheral blood of epithelial cancer patients. Nat Commun. Jan. 25 2019; 10 (1): 449. doi: 10.1038/s41467-019-08304-z
12. Lu Y C, Yao X, Crystal J S, et al. Efficient identification of mutated cancer antigens recognized by T cells associated with durable tumor regressions. Clin Cancer Res. Jul. 1 2014; 20 (13): 3401-10. doi: 10.1158/1078-0432.CCR-14-0433

1. A composition comprising a population of neoantigen-specific T cells that recognize one or more breast cancer-associated neoantigens.

2. The composition of claim 1, wherein the neoantigen-specific T cells recognize one or more breast cancer-associated neoantigens in TP53, AKT1, ESR1, PIK3CA, ERBB2, FRMPD3, GOLGA6L6, HISTIH2AE, MUC4, NBPF12, or SF3B1 genes.

3. The composition of claim 1 or 2, wherein;
(a) the neoantigen is from TP53 and is selected from the group consisting of TP53R175H; TP53 R248Q; TP53 R248W; TP53 R273C; and TP53 R273H;
(b) the neoantigen is from ESR1 and is selected from the group consisting of ESR1 K303R; ESR1 Y537S; and ESR1 D538G;
(c) the neoantigen is from AKT1 and is AKT1 E17K; and/or
(d) the neoantigen is from PIK3CA and is selected from the group consisting of PIK3CA E542K; PIK3CA E545K; PIK3CA H1047R; and PIK3CA H1047L.

4. (canceled)

5. (canceled)

6. (canceled)

7. (canceled)

8. (canceled)

9. (canceled)

10. (canceled)

11. The composition of claim 1, wherein the neoantigen-specific T cells are cultured ex vivo in the presence of two or more cytokines selected from the group consisting of IL-7, IL-12, IL-15 and IL-6.

12. The composition of claim 1, wherein the T-cell receptor of the T cells is encoded by DNA as follows:
(a) an alpha chain encoded by sequence comprising SEQ ID NO:4 or comprising at least 85% identity to SEQ ID NO:4; and a beta chain encoded by sequence comprising SEQ ID NO:5 or comprising at least 85% identity to SEQ ID NO:5;
(b) an alpha chain encoded by sequence comprising SEQ ID NO:6 or comprising at least 85% identity to SEQ ID NO:6; and a beta chain encoded by sequence comprising SEQ ID NO:7 or comprising at least 85% identity to SEQ ID NO:7;
(c) an alpha chain encoded by sequence comprising SEQ ID NO:8 or comprising at least 85% identity to SEQ ID NO:8; and a beta chain encoded by sequence comprising SEQ ID NO:9 or comprising at least 85% identity to SEQ ID NO:9; or
(d) an alpha chain encoded by sequence comprising SEQ ID NO:10 or comprising at least 85% identity to SEQ ID NO:10; and a beta chain encoded by sequence comprising SEQ ID NO:11 or comprising at least 85% identity to SEQ ID NO:11.

13. The composition of claim 1, wherein the T-cell receptor of the T cells comprises:
(a) an alpha chain comprising SEQ ID NO:12 or comprising at least 85% identity to SEQ ID NO: 12; and a beta chain comprising SEQ ID NO: 13 or comprising at least 85% identity to SEQ ID NO: 13;
(b) an alpha chain comprising SEQ ID NO:14 or comprising at least 85% identity to SEQ ID NO: 14; and a beta chain comprising SEQ ID NO: 15 or comprising at least 85% identity to SEQ ID NO: 15;
(c) an alpha chain comprising SEQ ID NO:16 or comprising at least 85% identity to SEQ ID NO: 16; and a beta chain comprising SEQ ID NO: 17 or comprising at least 85% identity to SEQ ID NO: 17; or
(d) an alpha chain comprising SEQ ID NO:18 or comprising at least 85% identity to SEQ ID NO: 18; and a beta chain comprising SEQ ID NO:19 or comprising at least 85% identity to SEQ ID NO: 19.

14. A composition comprising a polyclonal population of neoantigen-specific T cells that recognize one or more breast cancer-associated neoantigens and that recognize one or more other cancer antigens.

15. The composition of claim 14, wherein the one or more other antigens are breast cancer-associated antigens.

16. The composition of claim 14, wherein the one or more other antigens are not breast cancer-associated antigens.

17. The composition of 14, wherein the one or more other cancer antigens are neoantigens.

18. The composition of claim 17, wherein the neoantigens are associated with cancer other than breast cancer.

19. The composition of claim 14, wherein the neoantigen-specific T cells recognize one or more breast cancer-associated neoantigens in TP53, AKT1, ESR1, PIK3CA, ERBB2, FRMPD3, GOLGA6L6, HISTIH2AE, MUC4, NBPF12, or SF3B1 genes.

20. The composition of claim 5, wherein the neoantigen is AKT1 E17K; ESR1 K303R; ESR1 Y537S; ESR1 D538G; PIK3CA E542K; PIK3CA E545K; PIK3CA H1047R; PIK3CA H1047L; TP53 R175H; TP53 R248Q; TP53 R248W; TP53 R273C; TP53 R273H; ERBB2 L755S; ESR1 E380Q; ESR1 L536P; ESR1 S463P; ESR1 Y537C; ESR1 Y537N; EXOC4 S21L; FRMPD3 Q1757E; GOLGA6L6 M472I; HIST1H2AE K128M; MUC4 S2858P; NBPF12 E125Q; PIK3CA E453K; PIK3CA E726K; PIK3CA N345K; PIK3CA Q546K; SF3B1 K700E; TP53 G245S; TP53 H179R; TP53 H193R; TP53 1195T; TP53 R282W; or TP53 Y220C.

21. (canceled)

22. (canceled)

23. (canceled)

24. The composition of claim 1, wherein;
(a) the population comprises CD4+ T-lymphocytes and CD8+ T-lymphocytes;
(b) the neoantigen-specific T cells express αβ T cell receptors; and/or
(c) comprises MHC-restricted neoantigen-specific T cells.

25. (cancel)

26. (canceled)

27. The composition of claim 1, wherein the neoantigen-specific T cells comprise central memory and effector memory T cells.

28. The composition of claim 24, wherein the neoantigen-specific T cells are cultured ex vivo in the presence of two or more cytokines selected from the group consisting of IL-7, IL-12, IL-15 and IL-6.

29. The composition of claim 1, wherein:
(a) the neoantigen-specific T cells produce effector cytokines/molecules including IFN-gamma, TNF-alpha, GM-CSF, Granzyme-B, or perforin upon exposure to antigen;
(b) the neoantigen-specific T cells are able to lyse neoantigen-expressing target cells; and/or
(c) the neoantigen-specific T cells do not significantly lyse non-cancerous autologous or allogenic target cells.

30. (canceled)

31. (canceled)

32. A pharmaceutical composition comprising the composition of claim 1.

33. (canceled)

34. A composition comprising α chain and/or β chain T-cell receptor (TCR) polypeptides from the cells of claim 1.

35. (canceled)

36. (canceled)

37. (canceled)

38. (canceled)

39. (canceled)

40. A vector comprising α chain and/or β chain T-cell receptor (TCR) polypeptides from the cells of claim 1.

41. A cell engineered to express α chain and/or β chain T-cell receptor (TCR) polypeptides from the cells of claim 1.

42. (canceled)

43. (canceled)

44. The cell of claim 41, wherein the cell is engineered to comprise one or more further modifications.

45. The cell of claim 44, wherein the modifications comprise:
(a) disruption of one or more endogenous genes of the cells;
(b) one or more non-natural antigen-specific T cell receptors other than the engineered receptor comprising the α and/or β T-cell receptor (TCR) sequences;
(c) one or more cytokine receptors;
(d) one or more chimeric cytokine receptors;
(e) one or more cytokines; or
(f) a combination thereof.

46. The cell of claim 45, wherein the non-natural T cell receptors of (c) are TCRs directed against a tumor-associated antigen.

47. The cell of claim 45, wherein the neoantigen-specific T cells are modified to be directed against a tumor-associated antigen.

48. A method of lysing a target cell comprising contacting the target cell with the composition of claim 1.

49. (canceled)

50. (canceled)

51. A method of treating or preventing breast cancer in an individual in need thereof, comprising administering to the individual the composition of claim 1.

52. (canceled)

53. (canceled)

54. (canceled)

55. A method of treating or preventing breast cancer in an individual, comprising administering to the individual an effective amount of a population of neoantigen-specific T cells, wherein the neoantigen-specific T cells comprise a T cell receptor selected based on the ability to selectively bind a neoantigen in the cancer of the individual and wherein the neoantigen is HLA matched to the individual.

56. The method of claim 55, wherein the neoantigen-specific T cells are from a library of cell lines of neoantigen-specific T cells, wherein the T cell receptor is directed to a neoantigen that is known and the specific HLA polymorphism that presents the peptide to which the TCR binds is also known.

57. (canceled)

58. (canceled)

59. A method of producing neoantigen-specific T cells for treating or preventing breast cancer in an individual, comprising the steps of:
(a) obtaining or generating a library of cell lines of neoantigen-specific T cells, said step comprising one of the following:
(1) contacting PBMCs with one or more pepmix libraries, each pepmix library containing a plurality of overlapping peptides spanning all or at least a portion of one or more breast cancer-associated neoantigens;
(2) contacting T cells with APCs such as dendritic cells (DCs) primed with a plurality of pepmix libraries, each pepmix library containing a plurality of overlapping peptides spanning all or at least a portion of one or more breast cancer-associated neoantigens; or
(3) contacting T cells with APCs such as DCs nucleofected with at least one DNA plasmid encoding at least one breast cancer-associated neoantigen, or a portion thereof;
(b) screening cell lines from the library to select for cells that demonstrate specificity for the neoantigen, but not specificity for a corresponding wild-type sequence, and also identifying or having known HLA class restriction for the neoantigen;
(c) transfecting or transducing T cells with vectors expressing transgenic T-cell receptors from the cells in (b) that demonstrate specificity for the neoantigen; and
(d) administering to the individual an effective amount of the transduced or transfected T cells from (c) when the cancer of the individual comprises the neoantigen and when the individual is HLA matched for the neoantigen.

60. A method of treating or preventing breast cancer in an individual, comprising administering to the individual an effective amount of a population of neoantigen-specific T cells, wherein said T cells are generated by the following:
(a) producing a library of cell lines of neoantigen-specific T cells, said producing step comprising one of the following:
(1) contacting PBMCs with one or more pepmix libraries, each pepmix library containing a plurality of overlapping peptides spanning all or at least a portion of one or more breast cancer-associated neoantigens;
(2) contacting T cells with APCs such as dendritic cells (DCs) primed with a plurality of pepmix libraries, each pepmix library containing a plurality of overlapping peptides spanning all or at least a portion of one or more breast cancer-associated neoantigens; or
(3) contacting T cells with APCs such as DCs nucleofected with at least one DNA plasmid encoding at least one breast cancer-associated neoantigen, or a portion thereof;
(b) screening cell lines from the library to select for cells that demonstrate specificity for the neoantigen, but not a corresponding wild-type sequence, and also identifying HLA class restriction for the neoantigen;
(c) administering to the individual an effective amount of cells from the cell line when the cancer of the individual comprises the neoantigen and when the individual is HLA matched for the neoantigen.

61. The method of claim 60, wherein the neoantigen-specific T cells are cultured ex vivo in the presence of two or more cytokines selected from the group consisting of IL-7, IL-12, IL-15 and IL-6.

62. An engineered T cell receptor (TCR) comprising sequence encoded by DNA as follows:
(a) an alpha chain encoded by sequence comprising SEQ ID NO:4 or comprising at least 85% identity to SEQ ID NO:4; and a beta chain encoded by sequence comprising SEQ ID NO:5 or comprising at least 85% identity to SEQ ID NO:5;
(b) an alpha chain encoded by sequence comprising SEQ ID NO:6 or comprising at least 85% identity to SEQ ID NO:6; and a beta chain encoded by sequence comprising SEQ ID NO:7 or comprising at least 85% identity to SEQ ID NO:7;
(c) an alpha chain encoded by sequence comprising SEQ ID NO:8 or comprising at least 85% identity to SEQ ID NO:8; and a beta chain encoded by sequence comprising SEQ ID NO:9 or comprising at least 85% identity to SEQ ID NO:9; or
(d) an alpha chain encoded by sequence comprising SEQ ID NO:10 or comprising at least 85% identity to SEQ ID NO:10; and a beta chain encoded by sequence comprising SEQ ID NO: 11 or comprising at least 85% identity to SEQ ID NO:11.

63. An engineered T cell receptor (TCR) comprising:
(a) an alpha chain comprising SEQ ID NO:12 or comprising at least 85% identity to SEQ ID NO:12; and a beta chain comprising SEQ ID NO:13 or comprising at least 85% identity to SEQ ID NO:13;
(b) an alpha chain comprising SEQ ID NO:14 or comprising at least 85% identity to SEQ ID NO: 14; and a beta chain comprising SEQ ID NO: 15 or comprising at least 85% identity to SEQ ID NO:15;
(c) an alpha chain comprising SEQ ID NO:16 or comprising at least 85% identity to SEQ ID NO:16; and a beta chain comprising SEQ ID NO:17 or comprising at least 85% identity to SEQ ID NO: 17; or
(d) an alpha chain comprising SEQ ID NO:18 or comprising at least 85% identity to SEQ ID NO:18; and a beta chain comprising SEQ ID NO:19 or comprising at least 85% identity to SEQ ID NO:19.

64. A cell comprising one or more TCRs of claim 62.

65. (canceled)

66. (canceled)


SEQUENCE LISTING (28 sequences)

SEQ ID NO: 1 - protein, 15 aa, Homo sapiens
NVVPLYDLLLEMLDA

SEQ ID NO: 2 - protein, 15 aa, synthetic construct
NVVPLSDLLLEMLDA

SEQ ID NO: 3 - protein, 15 aa, synthetic construct
NVVPLYGLLLEMLDA

SEQ ID NO: 4 - DNA, 399 bp, synthetic construct
  source 1..399: mol_type = other DNA
atgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaacgaagatggaaggtttacagcacagctcaatagagccagccagtatatttccctgctcatcagagactccaagctcagtgattcagccacctacctctgtgcggtcctcgggtcaggaggaagctacatacctacatttggaagaggaaccagccttattgttcatccgtat

SEQ ID NO: 5 - DNA, 402 bp, synthetic construct
  source 1..402: mol_type = other DNA
atggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgcagaaggggcgcaagatacgcagtattttggcccaggcacccggctgacagtgctcgag

SEQ ID NO: 6 - DNA, 396 bp, synthetic construct
  source 1..396: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacgctcgataactatggtcagaattttgtctttggtcccggaaccagattgtccgtgctgccctat

SEQ ID NO: 7 - DNA, 408 bp, synthetic construct
  source 1..408: mol_type = other DNA
atgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtctagaaccgattggggatagtaatgaaaaactgttttttggcagtggaacccagctctctgtcttggag

SEQ ID NO: 8 - DNA, 384 bp, synthetic construct
  source 1..384: mol_type = other DNA
atgtggggagctttccttctctatgtttccatgaagatgggaggcactgcaggacaaagccttgagcagccctctgaagtgacagctgtggaaggagccattgtccagataaactgcatgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgtgagaggcgacgatggtcagaattttgtctttggtcccggaaccagattgtccgtgctgccctat

SEQ ID NO: 9 - DNA, 402 bp, synthetic construct
  source 1..402: mol_type = other DNA
atgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaagggaggggtgggaatcagccccagcattttggtgatgggactcgactctccatcctagag

SEQ ID NO: 10 - DNA, 405 bp, synthetic construct
  source 1..405: mol_type = other DNA
atgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggcgaccggtaaccagttctattttgggacagggacaagtttgacggtcattccaaat

SEQ ID NO: 11 - DNA, 402 bp, synthetic construct
  source 1..402: mol_type = other DNA
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagcttaggtgggccgtacaatgagcagttcttcgggccagggacacggctcaccgtgctagag

SEQ ID NO: 12 - protein, 133 aa, synthetic construct
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCAVLGSGGSYIPTFGRGTSLIVHPY

SEQ ID NO: 13 - protein, 134 aa, synthetic construct
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSAEGAQDTQYFGPGTRLTVLE

SEQ ID NO: 14 - protein, 132 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATLDNYGQNFVFGPGTRLSVLPY

SEQ ID NO: 15 - protein, 136 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLEPIGDSNEKLFFGSGTQLSVLE

SEQ ID NO: 16 - protein, 128 aa, synthetic construct
MWGAFLLYVSMKMGGTAGQSLEQPSEVTAVEGAIVQINCMYQTSGFYGLSWYQQHDGGAPTFLSYNALDGLEETGRFSSFLSRSDSYGYLLLQELQMKDSASYFCAVRGDDGQNFVFGPGTRLSVLPY

SEQ ID NO: 17 - protein, 134 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGRGGNQPQHFGDGTRLSILE

SEQ ID NO: 18 - protein, 135 aa, synthetic construct
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEATGNQFYFGTGTSLTVIPN

SEQ ID NO: 19 - protein, 134 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLGGPYNEQFFGPGTRLTVLE

SEQ ID NO: 20 - protein, 10 aa, synthetic construct
SDLLLEMLDA

SEQ ID NO: 21 - protein, 10 aa, synthetic construct
LSDLLLEMLD

SEQ ID NO: 22 - protein, 10 aa, synthetic construct
PLSDLLLEML

SEQ ID NO: 23 - protein, 10 aa, synthetic construct
VPLSDLLLEM

SEQ ID NO: 24 - protein, 10 aa, synthetic construct
VVPLSDLLLE

SEQ ID NO: 25 - protein, 10 aa, synthetic construct
NVVPLSDLLL

SEQ ID NO: 26 - protein, 9 aa, synthetic construct
VPLSDLLLE

SEQ ID NO: 27 - protein, 9 aa, synthetic construct
VVPLSDLLL

SEQ ID NO: 28 - protein, 9 aa, synthetic construct
NVVPLSDLL